SEQUENCE LISTING (30 sequences)

SEQ ID NO: 1 - DNA, 671 bp, Human Immunodeficiency Virus - 1
  CDS (12)...(662)
gatctgccaccatgggcggcaagtggtccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcctgctgcaccccatgtcccagcacggcatcgaggaccccgagaaggaggtgctggagtggaggttcgactccaagctggccttccaccacgtggccagggagctgcaccccgagtactacaaggactgctaaagcccgggc

SEQ ID NO: 2 - protein, 216 aa, Human Immunodeficiency Virus - 1
MGGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 3 - DNA, 719 bp, Human Immunodeficiency Virus - 1
  CDS (2)...(715)
catggatgcaatgaagagagggctctgctgtgtgctgctgctgtgtggagcagtcttcgtttcgcccagcgagatctcctccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcctgctgcaccccatgtcccagcacggcatcgaggaccccgagaaggaggtgctggagtggaggttcgactccaagctggccttccaccacgtggccagggagctgcaccccgagtactacaaggactgctaaagcc

SEQ ID NO: 4 - protein, 237 aa, Human Immunodeficiency Virus - 1
MDAMKRGLCCVLLLCGAVFVSPSEISSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCLLHPMSQHGIEDPEKEVLEWRFDSKLAFHHVARELHPEYYKDC

SEQ ID NO: 5 - DNA, 671 bp, Human Immunodeficiency Virus - 1
  CDS (12)...(662)
gatctgccaccatggccggcaagtggtccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcgccgcccaccccatgtcccagcacggcatcgaggaccccgagaaggaggtgctggagtggaggttcgactccaagctggccttccaccacgtggccagggagctgcaccccgagtactacaaggactgctaaagcccgggc

SEQ ID NO: 6 - protein, 217 aa, Human Immunodeficiency Virus - 1
MAGKWSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCAAHPMSQHGIEDPEKEVLEWRFDSKLAFHHVARELHPEYYKDCS

SEQ ID NO: 7 - DNA, 720 bp, Human Immunodeficiency Virus - 1
  CDS (2)...(715)
catggatgcaatgaagagagggctctgctgtgtgctgctgctgtgtggagcagtcttcgtttcgcccagcgagatctcctccaagaggtccgtgcccggctggtccaccgtgagggagaggatgaggagggccgagcccgccgccgacagggtgaggaggaccgagcccgccgccgtgggcgtgggcgccgtgtccagggacctggagaagcacggcgccatcacctcctccaacaccgccgccaccaacgccgactgcgcctggctggaggcccaggaggacgaggaggtgggcttccccgtgaggccccaggtgcccctgaggcccatgacctacaagggcgccgtggacctgtcccacttcctgaaggagaagggcggcctggagggcctgatccactcccagaagaggcaggacatcctggacctgtgggtgtaccacacccagggctacttccccgactggcagaactacacccccggccccggcatcaggttccccctgaccttcggctggtgcttcaagctggtgcccgtggagcccgagaaggtggaggaggccaacgagggcgagaacaactgcgccgcccaccccatgtcccagcacggcatcgaggaccccgagaaggaggtgctggagtggaggttcgactccaagctggccttccaccacgtggccagggagctgcaccccgagtactacaaggactgctaaagccc

SEQ ID NO: 8 - protein, 237 aa, Human Immunodeficiency Virus - 1
MDAMKRGLCCVLLLCGAVFVSPSEISSKRSVPGWSTVRERMRRAEPAADRVRRTEPAAVGVGAVSRDLEKHGAITSSNTAATNADCAWLEAQEDEEVGFPVRPQVPLRPMTYKGAVDLSHFLKEKGGLEGLIHSQKRQDILDLWVYHTQGYFPDWQNYTPGPGIRFPLTFGWCFKLVPVEPEKVEEANEGENNCAAHPMSQHGIEDPEKEVLEWRFDSKLAFHHVARELHPEYYKDC

SEQ ID NO: 9 - DNA, 4945 bp, E. coli
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatcaccatggatgcaatgaagagagggctctgctgtgtgctgctgctgtgtggagcagtcttcgtttcgcccagcgagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgctgcggccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 10 - DNA, 23 bp, Artificial Sequence
  oligonucleotide
ctatataagcagagctcgtttag

SEQ ID NO: 11 - DNA, 30 bp, Artificial Sequence
  oligonucleotide
gtagcaaagatctaaggacggtgactgcag

SEQ ID NO: 12 - DNA, 39 bp, Artificial Sequence
  oligonucleotide
gtatgtgtctgaaaatgagcgtggagattgggctcgcac

SEQ ID NO: 13 - DNA, 39 bp, Artificial Sequence
  oligonucleotide
gtgcgagcccaatctccacgctcattttcagacacatac

SEQ ID NO: 14 - DNA, 4432 bp, E. coli
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 15 - DNA, 4864 bp, E. coli
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 16 - DNA, 4867 bp, E. coli
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccgctgcggccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 78 bp, Artificial Sequence
  oligonucleotide
gatcaccatggatgcaatgaagagagggctctgctgtgtgctgctgctgtgtggagcagtcttcgtttcgcccagcga

SEQ ID NO: 18 - DNA, 78 bp, Artificial Sequence
  oligonucleotide
gatctcgctgggcgaaacgaagactgctccacacagcagcagcacacagcagagccctctcttcattgcatccatggt

SEQ ID NO: 19 - protein, 27 aa, Homo sapien
MDAMKRGLCCVLLLCGAVFVSPSEISS

SEQ ID NO: 20 - DNA, 33 bp, Artificial Sequence
  oligonucleotide
ggtacaaatattggctattggccattgcatacg

SEQ ID NO: 21 - DNA, 36 bp, Artificial Sequence
  oligonucleotide
ccacatctcgaggaaccgggtcaattcttcagcacc

SEQ ID NO: 22 - DNA, 38 bp, Artificial Sequence
  oligonucleotide
ggtacagatatcggaaagccacgttgtgtctcaaaatc

SEQ ID NO: 23 - DNA, 36 bp, Artificial Sequence
  oligonucleotide
cacatggatccgtaatgctctgccagtgttacaacc

SEQ ID NO: 24 - DNA, 39 bp, Artificial Sequence
  oligonucleotide
ggtacatgatcacgtagaaaagatcaaaggatcttcttg

SEQ ID NO: 25 - DNA, 35 bp, Artificial Sequence
  oligonucleotide
ccacatgtcgacccgtaaaaaggccgcgttgctgg

SEQ ID NO: 26 - DNA, 4864 bp, E. coli; identical to SEQ ID NO: 15

SEQ ID NO: 27 - DNA, 139 bp, E. coli / HIV-1
catgggtcttttctgcagtcaccgtccttgagatctgccaccatgggcggcaagtggtccaagaggtccgtgccccaccccgagtactacaaggactgctaaagcccgggcagatctgctgtgccttctagttgccagc

SEQ ID NO: 28 - DNA, 139 bp, E. coli / HIV-1
catgggtcttttctgcagtcaccgtccttgagatctgccaccatggccggcaagtggtccaagaggtccgtgccccaccccgagtactacaaggactgctaaagcccgggcagatctgctgtgccttctagttgccagc

SEQ ID NO: 29 - DNA, 203 bp, E. coli / HIV-1
catgggtcttttctgcagtcaccgtccttatatctagatcaccatggatgcaatgaagagagggctctgctgtgtgctgctgctgtgtggagcagtcttcgtttcgcccagcgagatctcctccaagaggtccgtgccccaccccgagtactacaaggactgctaaagcccgggcagatctgctgtgccttctagttgccagc

SEQ ID NO: 30 - DNA, 651 bp, Human Immunodificiency Virus - 1
atgggtggcaagtggtcaaaacgtagtgtgcctggatggtctactgtaagggaaagaatgagacgagctgagccagcagcagatagggtgagacgaactgagccagcagcagtaggggtgggagcagtatctcgagacctggaaaaacatggagcaatcacaagtagcaatacagcagctaccaatgctgattgtgcctggctagaagcacaagaggatgaggaagtgggttttccagtcagacctcaggtacctttaagaccaatgacttacaagggagctgtagatcttagccactttttaaaagaaaaggggggactggaagggctaattcactcacagaaaagacaagatatccttgatctgtgggtctaccacacacaaggctacttccctgattggcagaactacacaccagggccaggaatcagatttccattgacctttggatggtgcttcaagctagtaccagttgagccagaaaaggtagaagaggccaatgaaggagagaacaactgcttgttacaccctatgagccagcatgggatagaggacccggagaaggaagtgttagagtggaggtttgacagcaagctagcatttcatcacgtggcccgagagctgcatccggagtactacaaggactgctga

(19) United States
(12) Patent Application Publication
(10) Pub. No.: US 2005/0215508 A1
(43) Pub. Date: Sep. 29, 2005

(54) Polynucleotide vaccines expressing codon optimized HIV-1 Nef and modified HIV-1 Nef

(76) Inventors: John W. Shiver, Chalfont, PA (US); Xiaoping Liang, Eagleville, PA (US); Tong-Ming Fu, Lansdale, PA (US)

Correspondence Address: MERCK AND CO., INC, P O BOX 2000, RAHWAY, NJ, 07065-0907, US

(21) Appl. No.: 11/081,244

(22) Filed: Mar. 16, 2005

Related U.S. Application Data
(63) Continuation of application No. 10/149,640, filed on Jun. 13, 2002.
(60) Provisional application No. 60/172,442, filed on Dec. 17, 1999.

(51) Int. Cl. A61K 48/00; C07K 14/16

(52) U.S. Cl. 514/44; 530/350

(57) ABSTRACT

Pharmaceutical compositions which comprise HIV Nef DNA vaccines are disclosed, along with the production and use of these DNA vaccines. The nef-based DNA vaccines of the invention are administered directly introduced into living vertebrate tissue, preferably humans, and express the HIV Nef protein or biologically relevant portions thereof, inducing a cellular immune response which specifically recognizes human immunodeficiency virus-1 (HIV-1). The DNA molecules which comprise the open reading frame of these DNA vaccines are synthetic DNA molecules encoding codon optimized HIV-1 Nef and derivatives of optimized HIV-1 Nef, including nef modifications comprising amino terminal leader peptides, removal of the amino terminal myristylation site, and/or modification of the Nef dileucine motif. These modifications may effect wild type characteristics of Nef, such as myristylation and down regulation of host CD4.

### CROSS-REFERENCE TO RELATED APPLICATIONS

This application claims the benefit, under 35 U.S.C. §119(e), of U.S. provisional application 60/172,442, filed Dec. 17, 1999.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to HIV Nef polynucleotide pharmaceutical products, as well as the production and use thereof which, when directly introduced into living vertebrate tissue, preferably a mammalian host such as a human or a non-human mammal of commercial or domestic veterinary importance, express the HIV Nef protein or biologically relevant portions thereof within the animal, inducing a cellular immune response which specifically recognizes human immunodeficiency virus-1 (HIV-1). The polynucleotides of the present invention are synthetic DNA molecules encoding codon optimized HIV-1 Nef and derivatives of optimized HIV-1 Nef, including nef mutants which effect wild type characteristics of Nef, such as myristylation and down regulation of host CD4. The polynucleotide vaccines of the present invention should offer a prophylactic advantage to previously uninfected individuals and/or provide a therapeutic effect by reducing viral load levels within an infected individual, thus prolonging the asymptomatic phase of HIV-1 infection.

### BACKGROUND OF THE INVENTION

Human Immunodeficiency Virus-1 (HIV-1) is the etiological agent of acquired human immune deficiency syndrome (AIDS) and related disorders. HIV-1 is an RNA virus of the Retroviridae family and exhibits the 5′ LTR-gag-pol-env-LTR 3′organization of all retroviruses. The integrated form of HIV-1, known as the provirus, is approximately 9.8 Kb in length. Each end of the viral genome contains flanking sequences known as long terminal repeats (LTRs). The HIV genes encode at least nine proteins and are divided into three classes; the major structural proteins (Gag, Pol, and Env), the regulatory proteins (Tat and Rev); and the accessory proteins (Vpu, Vpr, Vif and Nef).

The gag gene encodes a 55-kilodalton (kDa) precursor protein (p55) which is expressed from the unspliced viral mRNA and is proteolytically processed by the HIV protease, a product of the pol gene. The mature p55 protein products are p17 (matrix), p24 (capsid), p9 (nucleocapsid) and p6.

The pol gene encodes proteins necessary for virus replication; a reverse transcriptase, a protease, integrase and RNAse H. These viral proteins are expressed as a Gag-Pol fusion protein, a 160 kDa precursor protein which is generated via a ribosomal frame shifting. The viral encoded protease proteolytically cleaves the Pol polypeptide away from the Gag-Pol fusion and further cleaves the Pol polypeptide to the mature proteins which provide protease (Pro, P10), reverse transcriptase (RT, P50), integrase (IN, p31) and RNAse H(RNAse, p15) activities.

The nef gene encodes an early accessory HIV protein (Nef) which has been shown to possess several activities such as down regulating CD4 expression, disturbing T-cell activation and stimulating HIV infectivity.

The env gene encodes the viral envelope glycoprotein that is translated as a 160-kilodalton (kDa) precursor (gp160) and then cleaved by a cellular protease to yield the external 120-kDa envelope glycoprotein (gp120) and the transmembrane 41-kDa envelope glycoprotein (gp41). Gp120 and gp41 remain associated and are displayed on the viral particles and the surface of HIV-infected cells.

The tat gene encodes a long form and a short form of the Tat protein, a RNA binding protein which is a transcriptional transactivator essential for HIV-1 replication.

The rev gene encodes the 13 kDa Rev protein, a RNA binding protein. The Rev protein binds to a region of the viral RNA termed the Rev response element (RRE). The Rev protein is promotes transfer of unspliced viral RNA from the nucleus to the cytoplasm. The Rev protein is required for HIV late gene expression and in turn, HIV replication.

Gp120 binds to the CD4/chemokine receptor present on the surface of helper T-lymphocytes, macrophages and other target cells in addition to other co-receptor molecules. X4 (macrophage tropic) virus show tropism for CD4/CXCR4 complexes while a R5 (T-cell line tropic) virus interacts with a CD4/CCR5 receptor complex. After gp120 binds to CD4, gp41 mediates the fusion event responsible for virus entry. The virus fuses with and enters the target cell, followed by reverse transcription of its single stranded RNA genome into the double-stranded DNA via a RNA dependent DNA polymerase. The viral DNA, known as provirus, enters the cell nucleus, where the viral DNA directs the production of new viral RNA within the nucleus, expression of early and late HIV viral proteins, and subsequently the production and cellular release of new virus particles. Recent advances in the ability to detect viral load within the host shows that the primary infection results in an extremely high generation and tissue distribution of the virus, followed by a steady state level of virus (albeit through a continual viral production and turnover during this phase), leading ultimately to another burst of virus load which leads to the onset of clinical AIDS. Productively infected cells have a half life of several days, whereas chronically or latently infected cells have a 3-week half life, followed by non-productively infected cells which have a long half life (over 100 days) but do not significantly contribute to day to day viral loads seen throughout the course of disease.

Destruction of CD4 helper T lymphocytes, which are critical to immune defense, is a major cause of the progressive immune dysfunction that is the hallmark of HIV infection. The loss of CD4 T-cells seriously impairs the body's ability to fight most invaders, but it has a particularly severe impact on the defenses against viruses, fungi, parasites and certain bacteria, including mycobacteria.

Effective treatment regimens for HIV-1 infected individuals have become available recently. However, these drugs will not have a significant impact on the disease in many parts of the world and they will have a minimal impact in halting the spread of infection within the human population. As is true of many other infectious diseases, a significant epidemiologic impact on the spread of HIV-1 infection will only occur subsequent to the development and introduction of an effective vaccine. There are a number of factors that have contributed to the lack of successful vaccine development to date. As noted above, it is now apparent that in a chronically infected person there exists constant virus production in spite of the presence of anti-HIV-1 humoral and cellular immune responses and destruction of virally infected cells. As in the case of other infectious diseases, the outcome of disease is the result of a balance between the kinetics and the magnitude of the immune response and the pathogen replicative rate and accessibility to the immune response. Pre-existing immunity may be more successful with an acute infection than an evolving immune response can be with an established infection. A second factor is the considerable genetic variability of the virus. Although anti-HIV-1 antibodies exist that can neutralize HIV-1 infectivity in cell culture, these antibodies are generally virus isolate-specific in their activity. It has proven impossible to define serological groupings of HIV-1 using traditional methods. Rather, the virus seems to define a serological “continuum” so that individual neutralizing antibody responses, at best, are effective against only a handful of viral variants. Given this latter observation, it would be useful to identify immunogens and related delivery technologies that are likely to elicit anti-HIV-1 cellular immune responses. It is known that in order to generate CTL responses antigen must be synthesized within or introduced into cells, subsequently processed into small peptides by the proteasome complex, and translocated into the endoplasmic reticulun/Golgi complex secretory pathway for eventual association with major histocompatibility complex (MHC) class I proteins. CD8⁺ T lymphocytes recognize antigen in association with class I MHC via the T cell receptor (TCR) and the CD8 cell surface protein. Activation of naive CD8⁺ T cells into activated effector or memory cells generally requires both TCR engagement of antigen as described above as well as engagement of costimulatory proteins. Optimal induction of CTL responses usually requires “help” in the form of cytokines from CD4⁺ T lymphocytes which recognize antigen associated with MHC class II molecules via TCR and CD4 engagement.

As introduced above, the nef gene encodes an early accessory HIV protein (Nef) which has been shown to possess several activities such as down regulating CD4 expression, disturbing T-cell activation and stimulating HIV infectivity. Zazopoulos and Haseltine (1992, Proc. Natl. Acad. Sci. 89: 6634-6638) disclose mutations to the HIV-1 nef gene which effect the rate of virus replication. The authors show that the nef open reading frame mutated to encode Ala-2 in place of Gly-2 inhibits myristolation of the protein and results in delayed viral replication rates in Jurkat cells and PBMCs.

Kaminchik et al. (1991, J. Virology 65 (2): 583-588) disclose an amino-terminal nef open reading frame mutated to encode Met-Ala-Ala in place of Met-Gly-Gly. The authors show that this mutant is deficient in myristolation.

Saksela et al. (1995, EMBO J. 14 (3): 484491) and Lee et al. (1995, EMBO J. 14 (20): 5006-5015) show the importance of a proline rich motif in HIV-1 Nef which mediates binding to a SH3 domain of the Hck protein. The authors conclude that this motif is important in the enhancement of viral replication but not down-regulation of CD4 expression.

Calarota et al. (1998, The Lancet 351: 1320-1325) present human clinical data concerning immunization of three HIV infected individuals with a DNA plasmid expressing wild type Nef. The authors conclude that immunization with a Nef encoding DNA plasmid induced a cellular immune response in the three individuals. However, two of the three patients were on alternative therapies during the study, and the authors conclude that the CTL response was most likely a boost to a pre-existing CTL response. In addition, the viral load increased substantially in two of the three patients during the course of the study.

Tobery et al. (1997, J. Exp. Med. 185 (5): 909-920) constructed two ubiquitin-nef (Ub-nef) fusion constructs, one which encoded the Nef initiating methionine and the other with an Arg residue at the amino terminus of the Nef open reading frame. The authors state that vaccinia- or plasmid-based immunization of mice with a Ub-nef construct containing an Arg residue at the amino terminus induces a Nef-specific CTL response. The authors suggest the expressed fusion protein is more efficiently presented to the MHC class I antigen presentation pathway, resulting in an improved cellular immune response.

Kim et al. (1997, J. Immunol. 158 (2): 816-826) disclose that co-administration of a plasmid DNA construct expressing IL-12 with a plasmid construct expressing Nef results in an improved cellular immune response in mice when compared to inoculation with the Nef construct alone. The authors reported a reduction in the humoral response from the Nef/IL-12 co-administration as compared to administration of the plasmid construct expressing Nef alone.

Moynier et al. (1998, Vaccine 16 (16): 1523-1530) show varying humoral responses in mice immunized with a DNA plasmid encoding Nef, depending upon the presence of absence of Freund's adjuvant. No data is disclosed regarding a cellular immune response in mice vaccinated with the aforementioned DNA construct alone.

Hanna et al. (1998, Cell 95:163-175) suggest that wild type Nef may play a critical role in AIDS pathogenicity.

It would be of great import in the battle against ADS to produce a prophylactic- and/or therapeutic-based HIV vaccine which generates a strong cellular immune response against an HIV infection. The present invention addresses and meets this needs by disclosing a class of DNA vaccines based on host delivery and expression of the early HIV gene, nef.

### SUMMARY OF THE INVENTION

The present invention relates to synthetic DNA molecules (also referred to herein as “polynucleotides”) and associated DNA vaccines (also referred to herein as “polynucleotide vaccines”) which elicit CTL responses upon administration to the host, such as a mammalian host and including primates and especially humans, as well as non-human mammals of commercial or domestic veterinary importance. The CTL-directed vaccines of the present invention should lower transmission rate to previously uninfected individuals and/or reduce levels of the viral loads within an infected individual, so as to prolong the asymptomatic phase of HIV-1 infection. In particular, the present invention relates to DNA vaccines which encode various forms of HIV-1 Nef, wherein administration, intracellular delivery and expression of the HIV-1 nef gene of interest elicits a host CTL and Th response. The preferred synthetic DNA molecules of the present invention encode codon optimized versions of wild type HIV-1 Nef, codon optimized versions of HIV-1 Nef fusion proteins, and codon optimized versions of HIV-1 Nef derivatives, including but not limited to nef modifications involving introduction of an amino-terminal leader sequence, removal of an amino-terminal myristylation site and/or introduction of dileucine motif mutations. The Nef-based fusion and modified proteins disclosed within this specification may possess altered trafficking and/or host cell function while retaining the ability to be properly presented to the host MHC I complex and in turn elicit a host CTL and Th response.

A particular embodiment of the present invention relates to a DNA molecule encoding HIV-1 Nef from the HIV-1 jfrl isolate wherein the codons are optimized for expression in a mammalian system such as a human. The DNA molecule which encodes this protein is disclosed herein as SEQ ID NO:1, while the expressed open reading frame is disclosed herein as SEQ ID NO:2.

In another embodiment of the present invention, a codon optimized DNA molecule encoding a protein containing the human plasminogen activator (tpa) leader peptide fused with the NH₂-terminus of the HIV-1 Nef polypeptide. The DNA molecule which encodes this protein is disclosed herein as SEQ ID NO:3, while the expressed open reading frame is disclosed herein as SEQ ID NO:4.

In an additional embodiment, the present invention relates to a DNA molecule encoding optimized HIV-1 Nef wherein the open reading frame codes for modifications at the amino terminal myristylation site (Gly-2 to Ala-2) and substitution of the Leu-174-Leu-175 dileucine motif to Ala-174-Ala-175, herein described as opt nef (G2A,LLAA). The DNA molecule which encodes this protein is disclosed herein as SEQ ID NO:5, while the expressed open reading frame is disclosed herein as SEQ ID NO:6.

Another additional embodiment of the present invention relates to a DNA molecule encoding optimized HIV-1 Nef wherein the amino terminal myristylation site and dileucine motif have been deleted, as well as comprising a tPA leader peptide. This DNA molecule, opt tpanef (LLAA), comprises an open reading frame which encodes a Nef protein containing a tPA leader sequence fused to amino acid residue 6-216 of HIV-1 Nef (jfrl), wherein Leu-174 and Leu-175 are substituted with Ala-174 and Ala-175, herein referred to as opt tpanef (LLAA) is disclosed herein as SEQ ID NO:7, while the expressed open reading frame is disclosed herein as SEQ ID NO:8.

The present invention also relates to non-codon optimized versions of DNA molecules and associated DNA vaccines which encode the various wild type and modified forms of the HIV Nef protein disclosed herein. Partial or fully codon optimized DNA vaccine expression vector constructs are preferred, but it is within the scope of the present invention to utilize “non-codon optimized” versions of the constructs disclosed herein, especially modified versions of HIV Nef which are shown to promote a substantial cellular immune response subsequent to host administration.

The DNA backbone of the DNA vaccines of the present invention are preferably DNA plasmid expression vectors. DNA plasmid expression vectors utilized in the present invention include but are not limited to constructs which comprise the cytomegalovirus promoter with the intron A sequence (CMV-intA) and a bovine growth hormone transcription termination sequence. In addition, the DNA plasmid vectors of the present invention preferably comprise an antibiotic resistance marker, including but not limited to an ampicillin resistance gene, a neomycin resistance gene or any other pharmaceutically acceptable antibiotic resistance marker. In addition, an appropriate polylinker cloning site and a prokaryotic origin of replication sequence are also preferred. Specific DNA vectors of the present invention include but are not limited to V1, V1J (SEQ ID NO:14), V1Jneo (SEQ ID NO:15), V1Jns (FIG. 1A, SEQ ID NO:16), V1R (SEQ ID NO:26), and any of the aforementioned vectors wherein a nucleotide sequence encoding a leader peptide, preferably the human tPA leader, is fused directly downstream of the CMV-intA promoter, including but not limited to V1Jns-tpa, as shown in FIG. 1B and SEQ ID NO:19.

The present invention especially relates to a DNA vaccine and a pharmaceutically active vaccine composition which contains this DNA vaccine, and the use as a prophylactic and/or therapeutic vaccine for host immunization, preferably human host immunization, against an HIV infection or to combat an existing HIV condition. These DNA vaccines are represented by codon optimized DNA molecules encoding HIV-1 Nef of biologically active Nef modifications or Nef-containing fusion proteins which are ligated within an appropriate DNA plasmid vector, with or without a nucleotide sequence encoding a functional leader peptide. DNA vaccines of the present invention relate in part to codon optimized DNA molecules encoding HIV-1 Nef of biologically active Nef modifications or Nef-containing fusion proteins ligated in DNA vectors V1, V1J (SEQ ID NO:14), V1Jneo (SEQ ID NO:15), V1Jns (FIG. 1A, SEQ ID NO:16), V1R (SEQ ID NO:26), or any of the aforementioned vectors wherein a nucleotide sequence encoding a leader peptide, preferably the human tPA leader, is fused directly downstream of the CMV-intA promoter, including but not limited to V1Jns-tpa, as shown in FIG. 1B and SEQ ID NO:19. Especially preferred DNA vaccines of the present invention include codon optimized DNA vaccine constructs V1Jns/nef, V1Jns/tpanef, V1Jns/tpanef(LLAA) and V1Jns/(G2A,LLAA), as exemplified in Example Section 2.

The present invention also relates to HIV Nef polynucleotide pharmaceutical products, as well as the production and use thereof, wherein the DNA vaccines are formulated with an adjuvant or adjuvants which may increase immunogenicity of the DNA polynucleotide vaccines of the present invention, namely by increasing a humoral response to inoculation. A preferred adjuvant is an aluminum phosphate-based adjuvant or a calcium phosphate based adjuvant, with an aluminum phosphate adjuvant being especially preferred. Another preferred adjuvant is a non-ionic block copolymer, preferably comprising the blocks of polyoxyethylene (POE) and polyoxypropylene (POP) such as a POE-POP-POE block copolymer. These adjuvanted forms comprising the DNA vaccines disclosed herein are useful in increasing humoral responses to DNA vaccination without imparting a negative effect on an appropriate cellular immune response.

As used herein, a DNA vaccine or DNA polynucleotide vaccine or polynucleotide vaccine is a DNA molecule (i.e., “nucleic acid”, “polynucleotide”) which contains essential regulatory elements such that upon introduction into a living, vertebrate cell, it is able to direct the cellular machinery to produce translation products encoded by the respective nef genes of the present invention.

### BRIEF DESCRIPTION OF THE FIGURES

FIG. 1A-B show a schematic representation of DNA vaccine expression vectors V1Jns (A) and V1Jns/tpa utilized for HIV-1 nef and HIV-1 modified nef constructs.

FIG. 2A-B show a nucleotide sequence comparison between wild type nef(jrfl) and codon optimized nef. The wild type nef gene from the jrfl isolate consists of 648 nucleotides capable of encoding a 216 amino acid polypeptide. WT, wild type sequence (SEQ ID NO:9); opt, codon-optimized sequence (contained within SEQ ID NO:1). The Nef amino acid sequence is shown in one-letter code (SEQ ID NO:2).

FIG. 3A-C show nucleotide sequences at junctions between nef coding sequence and plasmid backbone of nef expression vectors V1Jns/nef (FIG. 3A), V1Jns/nef(G2A,LLAA) (FIG. 3B), V1Jns/tpanef (FIG. 3C) and V1Jns/tpanef(LLAA) (FIG. 3C, also). 5′ and 3′ flanking sequences of codon optimized nef or codon optimized nef mutant genes are indicated by bold/italic letters; nef and nef mutant coding sequences are indicated by plain letters. Also indicated (as underlined) are the restriction endonuclease sites involved in construction of respective nef expression vectors. V1Jns/tpanef and V1Jns/tpanef(LLAA) have identical sequences at the junctions.

FIG. 4 shows a schematic presentation of nef and nef derivatives. Amino acid residues involved in Nef derivatives are presented. Glycine 2 and Leucine 174 and 175 are the sites involved in myristylation and dileucine motif, respectively. For both versions of the tpanef fusion genes, the putative leader peptide cleavage sites are indicated with “*”, and a exogenous serine residue introduced during the construction of the mutants is underlined.

FIG. 5 shows Western blot analysis of nef and modified nef proteins expressed in transfected 293 cells. 293 cells grown in 100 mm culture dish were transfected with respective codon optimized nef constructs. Sixty hours post transfection, supernatant and cells were collected separately and separated on 10% SDS-PAGE under reducing conditions. The proteins were transferred into a PVDF membrane and probed with a mixture of Gag mAb and Nef mAbs, both at 1:2000 dilution. The protein signals were detected with ECL. (A) cells transfected with V1Jns/gag only; (B) cells transfected with V1Jns/gag and V1Jns/nef; (C) cells transfected with V1Jns/gag and V1Jns/nef(G2A, LLAA); (D) cells transfected with V1Jns/gag and V1Jns/tpanef; (E) cells transfected with V1Jns/gag and V1Jns/tpanef(LLAA). The low case letter c and m represent medium and cellular fractions, respectively. M.W.=molecular weight marker.

FIG. 6 shows an Elispot assay of cell-mediated responses to Nef peptides. Three strains of mice, Balb/c, C57B/6 and C3H, were immunized with 50 mcg of V1Jns/nef (codon optimized) and boosted twice with a two-week interval. Two weeks following the final immunization, splenocytes were isolated and tested in an Elispot assay against respective Nef peptide pools. As a control, splenocytes were from non-immunized naive mice were tested in parallel. Nef peptide pool A consists of all 21 Nef peptides; Nef peptide pool B consists of 11 non-overlapping peptide started from residue 1; Nef peptide pool C consists of 10 non-overlapping peptides started from residue 11. SFC, INF-gamma secreting spot-forming cells.

FIG. 7A-C show Nef-specific CD8 and CD4 epitope mapping. The immunization regime is as per FIG. 6. Mouse splenocytes were isolated and fractionated into CD8⁺ and CD8⁻ cells using Miltenyi's magnetic cell separator. The resultant CD8⁺ and CD8⁻ cells were then tested in an Elispot assay against individual Nef peptides. SFC, INF-gamma secreting spot-forming cells. The mice strains tested are Balb/c mice (FIG. 7A), C57B/6 mice (FIG. 7B), and C3H mice (FIG. 7C).

FIG. 8A-C show identification of a Nef CTL epitope. Splenocytes from nef immunized C57B/6 mice were stimulated in vitro with peptide-pulsed, irradiated naïve splenocytes for 7 days. Following the in vitro stimulation, cells were harvested and tested in a standard ⁵¹Cr-releasing assay using peptide pulsed EL-4 cells as targets. Open symbol, specific killings of EL-4 cells without peptide; solid symbol, specific killing of EL-4 cells with peptide. Panel A—peptide Nef 51-70; Panel B—peptide Nef 60-68, Panel C—peptide Nef 58-70.

FIG. 9A-B shows a comparison of the immunogenicity of codon optimized DNA vaccine vectors expressing Nef and modified forms of Nef C57BL/6 mice, five per group, were immunized with 100 mcg of the indicated nef constructs. Fourteen days following immunization, splenocytes were collected and tested against the Nef CD8 (aa58-66) and CD4 (aa81-100) peptides. Identical immunization regimens were used for both experiments. In experiment 1 (Panel A), three codon optimized nef constructs were tested, namely, V1Jns/nef, V1Jns/tpanef(LLAA) and V1Jns/nef(G2A,LLAA), whereas in experiment 2 (Panel B) all four codon optimized nef constructs were tested. The data represent means plus standard deviation of 5 mice per group.

### DETAILED DESCRIPTION OF THE INVENTION

The present invention relates to synthetic DNA molecules (also referred to herein as “nucleic acid” molecules or “polynucleotides”) and associated DNA vector vaccines (also referred to herein as “polynucleotide vaccines”) which elicit CTL and humoral responses upon administration to the host, including primates and especially humans. In particular, the present invention relates to DNA vector vaccines which encode various forms of HIV-1 Nef, wherein administration, intracellular delivery and expression of the HIV-1 nef gene of interest elicits a host CTL and Th response. The synthetic DNA molecules of the present invention encode codon optimized versions of wild type HIV-1 Nef, codon optimized versions of HIV-1 Nef fusion proteins, and codon optimized versions of HIV-1 Nef derivatives, including but not limited to nef modifications involving introduction of an amino-terminal leader sequence, removal of an amino-terminal myristylation site and/or introduction of dileucine motif mutations. In some instances the Nef-based fusion and modified proteins disclosed within this specification possess altered trafficking and/or host cell function while retaining the ability to be properly presented to the host MHC I complex. Those skilled in the art will recognize that the use of nef genes from HIV-2 strains which express Nef proteins having analogous function to HIV-1 Nef would be expected to generate immune responses analogous to those described herein for HIV-1 constructs.

In order to generate a CTL response, the immunogen must be synthesized within (MHCI presentation) or introduced into cells (MHCII presentation). For intracellular synthesized immunogens, the protein is expressed and then processed into small peptides by the proteasome complex, and translocated into the endoplasmic reticulum/Golgi complex secretory pathway for eventual association with major histocompatibility complex (MHC) class I proteins. CD8⁺ T lymphocytes recognize antigen in association with class I MHC via the T cell receptor (TCR). Activation of naive CD8⁺ T cells into activated effector or memory cells generally requires both TCR engagement of antigen as described above as well as engagement of co-stimulatory proteins. Optimal induction of CTL responses usually requires “help” in the form of cytokines from CD4⁺ T lymphocytes which recognize antigen associated with MHC class II molecules via TCR.

The HIV-1 genome employs predominantly uncommon codons compared to highly expressed human genes. Therefore, the nef open reading frame has been synthetically manipulated using optimal codons for human expression. As noted above, a preferred embodiment of the present invention relates to DNA molecules which comprise a HIV-1 nef open reading frame, whether encoding full length nef or a modification or fusion as described herein, wherein the codon usage has been optimized for expression in a mammal, especially a human.

In a particular embodiment of the present invention, a DNA molecule encoding HIV-1 Nef from the HIV-1 jfrl isolate wherein the codons are optimized for expression in a mammalian system such as a human. The nucleotide sequence of the codon optimized version of HIV-1 jrfl nef gene is disclosed herein as SEQ ID NO:1, as shown herein:

(SEQ ID NO:1)
GATCTGCCAC CATGGGCGGC AAGTGGTCCA AGAGGTCCGT GCCCGGCTGC TCCACCGTGA
GGGAGAGGAT GAGGAGGGCC GAGCCCGCCG CCGACAGGGT GAGGAGGACC GAGCCCGCCG
CCGTGGGCGT GGGCGCCGTG TCCAGGGACC TGGAGAAGCA CGGCGCCATC ACCTCCTCCA
ACACCGCCGC CACCAACGCC GACTGCGCCT GGCTGGAGGC CCAGGAGGAC GAGGAGGTGG
GCTTCCCCGT GAGGOOCCAG GTGCCCCTGA GGCCCATGAC CTACAAGGGC GCCGTGGACC
TGTCCCACTT CCTGAAGGAG AAGGGCGGCC TGGAGGGCCT GATCCACTCC CAGAAGAGGC
AGGACATCCT GGACCTGTGG GTGTACCACA CCCAGGGCTA CTTCCCCGAC TGGCAGAACT
ACACCCCCGG CCCCGGCATC AGGTTCCCCC TGACCTTCGG CTGGTGCTTC AAGCTGGTGC
CCGTGGAGCC CGAGAAGGTG CAGGAGGCCA ACGAGGGCGA GAACAACTGC CTGCTGCACC
CCATGTCCCA GCACGGCATC GAGGACCCCG AGAAGGAGGT CCTGGAGTGG AGGTTCGACT
CCAAGCTGGC CTTCCACCAC GTGGCCAGGG AGCTGCACCC CGAGTACTAC AAGGACTGCT
AAAGCCCGGG C.

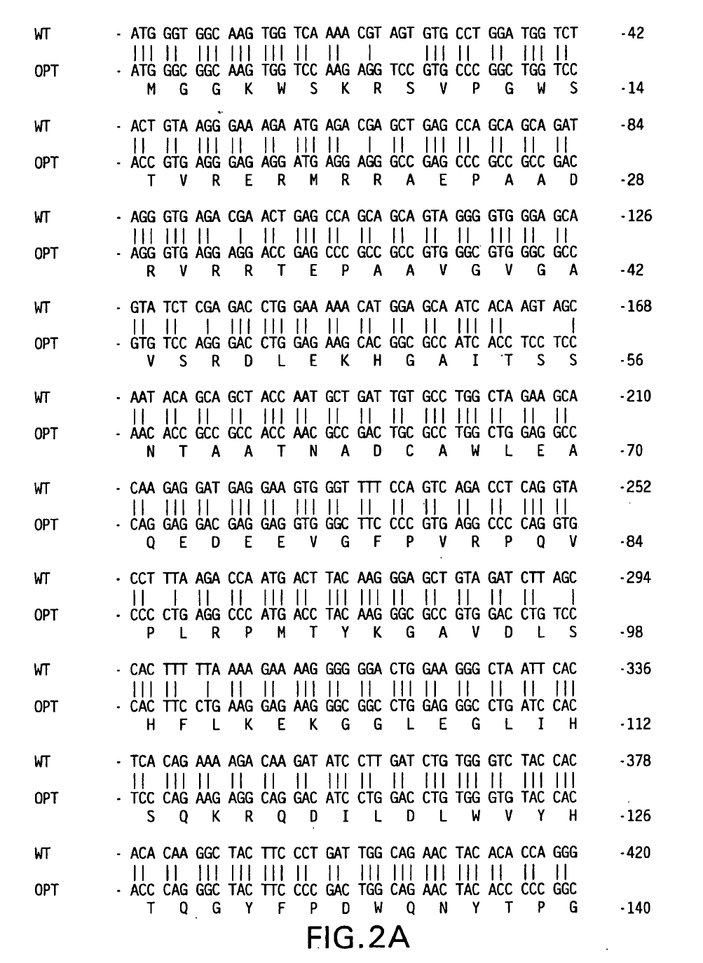

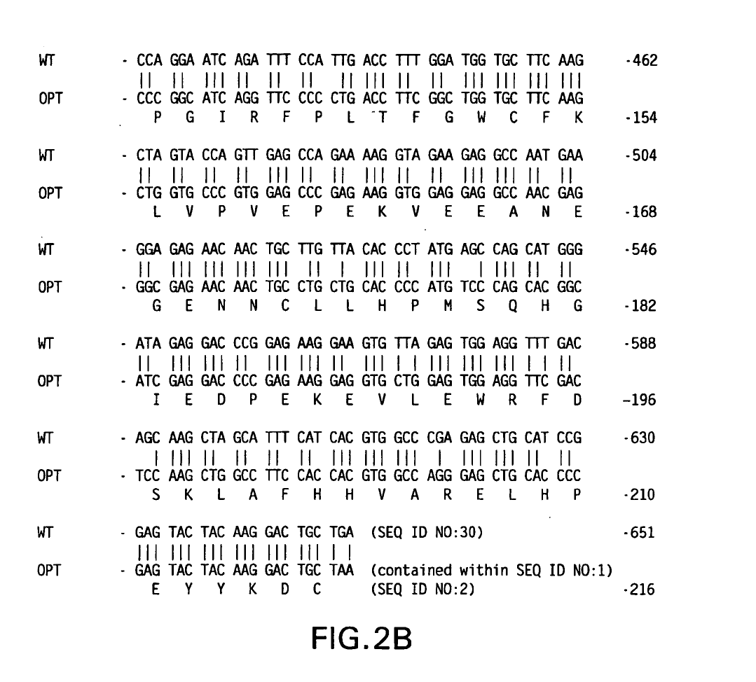

As can be discerned from comparing native to optimized codon usage in FIG. 2A-B, the following codon usage for mammalian optimization is preferred: Met (ATG), Gly (GGC), Lys (AAG), Trp (TGG), Ser (TCC), Arg (AGG), Val (GTG), Pro (CCC), Thr (ACC), Glu (GAG); Leu (CTG), His (CAC), Ile (ATC), Asn (AAC), Cys (TGC), Ala (GCC), Gln (CAG), Phe (ITC) and Tyr (TAC). For an additional discussion relating to mammalian (human) codon optimization, see WO 97/31115 (PCT/US97/02294), which is hereby incorporated by reference.

The open reading frame for SEQ ID NO:1 above comprises an initiating methionine residue at nucleotides 12-14 and a “TAA” stop codon from nucleotides 660-662. The open reading frame of SEQ ID NO: 1 provides for a 216 amino acid HIV-1 Nef protein expressed through utilization of a codon optimized DNA vaccine vector. The 216 amino acid HIV-1 Nef(jfrl) protein is disclosed herein as SEQ ID NO:2, and as follows:

(SEQ ID NO:2)
Met Gly Gly Lys Trp Ser Lys Arg Ser Val Pro Gly Trp Ser Thr Val
Arg Glu Arg Met Arg Arg Ala Glu Pro Ala Ala Asp Arg Val Arg Arg
Thr Glu Pro Ala Ala Val Gly Val Gly Ala Val Ser Arg Asp Leu Glu
Lys His Gly Ala Ile Thr Ser Ser Asn Thr Ala Ala Thr Asn Ala Asp
Cys Ala Trp Leu Glu Ala Gln Glu Asp Glu Glu Val Gly Phe Pro Val
Arg Pro Gln Val Pro Leu Arg Pro Met Thr Tyr Lys Gly Ala Val Asp
Leu Ser His Phe Leu Lys Glu Lys Gly Gly Leu Glu Gly Leu Ile His
Ser Gln Lys Arg Gln Asp Ile Leu Asp Leu Trp Val Tyr His Thr Gln
Gly Tyr Phe Pro Asp Trp Gln Asn Tyr Thr Pro Gly Pro Gly Ile Arg
Phe Pro Leu Thr Phe Gly Trp Cys Phe Lys Leu Val Pro Val Glu Pro
Glu Lys Val Glu Glu Ala Asn Glu Gly Glu Asn Asn Cys Leu Leu His
Pro Met Ser Gln His Gly Ile Glu Asp Pro Glu Lys Glu Val Leu Glu
Trp Arg Phe Asp Ser Lys Leu Ala Phe His His Val Ala Arg Glu Leu
His Pro Glu Tyr Tyr Lys Asp Cys.

HIV-1 Nef is a 206 amino acid cytosolic protein which associates with the inner surface of the host cell plasma membrane through myristylation of Gly-2 (Franchini et al., 1986, Virology 155: 593-599). While not all possible Nef functions have been elucidated, it has become clear that correct trafficking of Nef to the inner plasma membrane promotes viral replication by altering the host intracellular environment to facilitate the early phase of the HIV-1 life cycle and by increasing the infectivity of progeny viral particles. In one aspect of the invention regarding codon-optimized, protein-modified polypeptides, either the DNA vaccine vector molecule or the HIV-1 nef construct is modified to contain a nucleotide sequence which encodes a heterologous leader peptide such that the amino terminal region of the expressed protein will contain the leader peptide. The diversity of function that typifies eukaryotic cells depends upon the structural differentiation of their membrane boundaries. To generate and maintain these structures, proteins must be transported from their site of synthesis in the endoplasmic reticulum to predetermined destinations throughout the cell. This requires that the trafficking proteins display sorting signals that are recognized by the molecular machinery responsible for route selection located at the access points to the main trafficking pathways. Sorting decisions for most proteins need to be made only once as they traverse their biosynthetic pathways since their final destination, the cellular location at which they perform their function, becomes their permanent residence. Maintenance of intracellular integrity depends in part on the selective sorting and accurate transport of proteins to their correct destinations. Defined sequence motifs exist in proteins which can act as ‘address labels’. A number of sorting signals have been found associated with the cytoplasmic domains of membrane proteins. An effective induction of CTL responses often required sustained, high level endogenous expression of an antigen. In light of its diverse biological activities, vaccines composed of wild-type Nef could potentially have adverse effects on the host cells. As membrane-association via myristylation is an essential requirement for most of Nef's function, mutants lacking myristylation, by glycine-to-alanine change, change of the dileucine motif and/or by substitution with a tpa leader sequence as described herein, will be functionally defective, and therefore will have improved safety profile compared to wild-type Nef for use as an HIV-1 vaccine component.

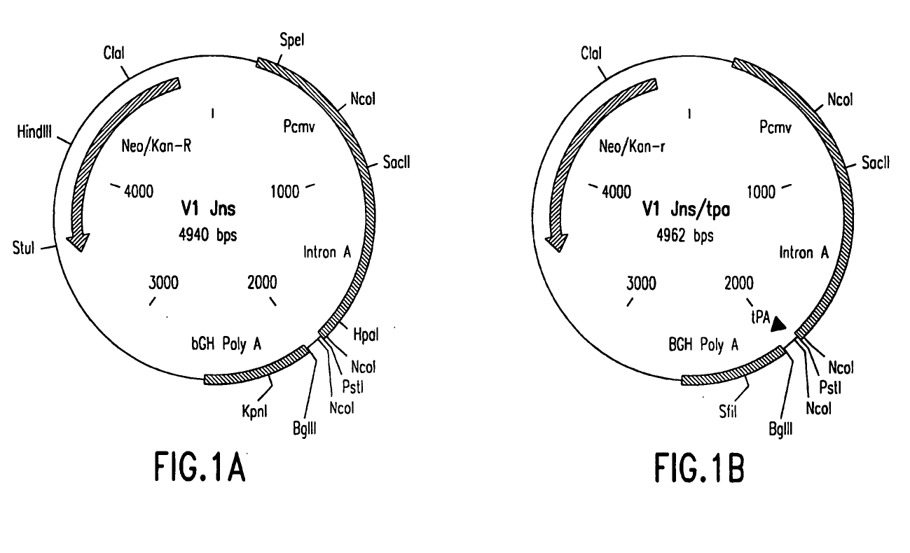

In a preferred and exemplified embodiment of this portion of the invention, either the DNA vector or the HIV-1 nef nucleotide sequence is modified to include the human tissue-specific plasminogen activator (tPA) leader. As shown in FIG. 1A-B for the DNA vector V1Jns, a DNA vector which may be utilized to practice the present invention may be modified by known recombinant DNA methodology to contain a leader signal peptide of interest, such that downstream cloning of the modified HIV-1 protein of interest results in a nucleotide sequence which encodes a modified HIV-1 tPA/Nef protein. In the alternative, as noted above, insertion of a nucleotide sequence which encodes a leader peptide may be inserted into a DNA vector housing the open reading frame for the Nef protein of interest. Regardless of the cloning strategy, the end result is a polynucleotide vaccine which comprises vector components for effective gene expression in conjunction with nucleotide sequences which encode a modified HIV-1 Nef protein of interest, including but not limited to a HIV-1 Nef protein which contains a leader peptide. The amino acid sequence of the human tPA leader utilized herein is as follows:

| | |
|---|---|
| MDAMKRGLCCVLLLCGAVFVSPSEISS. | (SEQ ID NO:19) |

It has been shown that myristylation of Gly-2 in conjunction with a dileucine motif in the carboxy region of the protein is essential for Nef-induced down regulation of CD4 (Aiken et al., 1994, Cell 76: 853-864) via endocytosis. It has also been shown that Nef expression promotes down regulation of MHCI (Schwartz et al., 1996, Nature Medicine 2 (3): 338-342) via endocytosis. The present invention relates in part to DNA vaccines which encode modified Nef proteins altered in trafficking and/or functional properties. The modifications introduced into the DNA vaccines of the present invention include but are not limited to additions, deletions or substitutions to the nef open reading frame which results in the expression of a modified Nef protein which includes an amino terminal leader peptide, modification or deletion of the amino terminal myristylation site, and modification or deletion of the dileucine motif within the Nef protein and which alter function within the infected host cell. Therefore, a central theme of the DNA molecules and DNA vaccines of the present invention is (1) host administration and intracellular delivery of a codon optimized nef-based DNA vector vaccine; (2) expression of a modified Nef protein which is immunogenic in terms of eliciting both CTL and Th responses; and, (3) inhibiting or at least altering known early viral functions of Nef which have been shown to promote HIV-1 replication and load within an infected host.

In another preferred and exemplified embodiment of the present invention, the nef coding region is altered, resulting in a DNA vaccine which expresses a modified Nef protein wherein the amino terminal Gly-2 myristylation residue is either deleted or modified to express alternate amino acid residues.

In another preferred and exemplified embodiment of the present invention, the nef coding region is altered, resulting in a DNA vaccine which expresses a modified Nef protein wherein the di leucine motif is either deleted or modified to express alternate amino acid residues.

Therefore, the present invention relates to an isolated DNA molecule, regardless of codon usage, which expresses a wild type or modified Nef protein as described herein, including but not limited to modified Nef proteins which comprise a deletion or substitution of Gly 2, a deletion or substitution of Leu 174 and Leu 175 and/or inclusion of a leader sequence.

The present invention also relates to a substantially purified protein expressed from the DNA polynucleotide vaccines of the present invention, especially the purified proteins set forth below as SEQ ID NOs: 2, 4, 6, and 8. These purified proteins may be useful as protein-based HIV vaccines.

In a specific embodiment of the invention as it relates DNA vaccines encoding modified forms of HIV-1, an open reading frame which encodes a Nef protein which comprises a tPA leader sequence fused to amino acid residue 6-216 of HIV-1 Nef (jfrl) is referred to herein as opt tpanef. The nucleotide sequence comprising the open reading frame of opt tpanef is disclosed herein as SEQ ID NO:3, as shown below:

(SEQ ID NO:3)
CATGGATGCA ATGAAGAGAG GGCTCTGCTG TGTGCTGCTG CTGTGTGGAG CAGTCTTCGT
TTCGCCCAGC GAGATCTCCT CCAAGAGGTC CGTGCCCGGC TGGTCCACCG TGAGGGAGAG
GATGAGGAGG GCCGAGCCCG CCGCCGACAG GGTGAGGAGG ACCGAGCCCG CCGCCGTGGG
CGTGGGCGCC GTGTCCAGGG ACCTGGAGAA GCACGGCGCC ATCACCTCCT CCAACACCGC
CGCCACCAAC GCCGACTGCG CCTGGCTGGA GGCCCAGGAG GACGAGGAGG TGGGCTTCCC
CGTGAGGCCC CAGGTGCCCC TGAGGCCCAT GACCTACAAG GGCGCCGTGG ACCTGTCCCA
CTTCCTGAAG GAGAAGGGCG GCCTGGAGGG CCTGATCCAC TCCCAGAAGA GGCAGGACAT
CCTGCACCTG TGGGTGTACC ACACCCAGGG CTACTTCCCC GACTGGCAGA ACTACACCCC
CGGCCCCGGC ATCAGGTTCC CCCTGACCTT CGGCTGGTGC TTCAAGCTGG TGCCCGTGGA
GCCCGAGAAG GTGGAGGAGC CCAACGAGGG CGAGAACAAC TGCCTGCTGC ACCCCATGTC
CCACCACGGC ATCGAGGACC CCGAGAAGGA GGTGCTGGAG TGGAGGTTCG ACTCCAAGCT
GGCCTTCCAC CACGTGGCCA GGGAGCTGCA CCCCGAGTAC TACAAGGACT GCTAAAGCC.

The open reading frame for SEQ ID NO:3 comprises an initiating methionine residue at nucleotides 2-4 and a “TAA” stop codon from nucleotides 713-715. The open reading frame of SEQ ID NO:3 provides for a 237 amino acid HIV-1 Nef protein which comprises a tPA leader sequence fused to amino acids 6-216 of HIV-1 Nef, including the dileucine motif at amino acid residues 174 and 175. This 237 amino acid tPA/Nef (jfrl) fusion protein is disclosed herein as SEQ ID NO:4, and is shown as follows:

(SEQ ID NO:4)
Met Asp Ala Met Lys Arg Gly Leu Cys Cys Val Leu Leu Leu Cys Gly
Ala Val Phe Val Ser Pro Ser Glu Ile Ser Ser Lys Arg Ser Val Pro
Gly Trp Ser Thr Val Arg Glu Arg Met Arg Arg Ala Glu Pro Ala Ala
Asp Arg Val Arg Arg Thr Glu Pro Ala Ala Val Gly Val Gly Ala Val
Ser Arg Asp Leu Glu Lys His Gly Ala Ile Thr Ser Ser Asn Thr Ala
Ala Thr Asn Ala Asp Cys Ala Trp Leu Glu Ala Gln Glu Asp Glu Glu
Val Gly Phe Pro Val Arg Pro Gln Val Pro Leu Arg Pro Met Thr Tyr
Lys Gly Ala Val Asp Leu Ser His Phe Leu Lys Glu Lys Gly Gly Leu
Glu Gly Leu Ile His Ser Gln Lys Arg Gln Asp Ile Leu Asp Leu Trp
Val Tyr His Thr Gln Gly Tyr Phe Pro Asp Trp Gln Asn Tyr Thr Pro
Gly Pro Gly Ile Arg Phe Pro Leu Thr Phe Gly Trp Cys Phe Lys Leu
Val Pro Val Glu Pro Glu Lys Val Glu Glu Ala Asn Glu Gly Glu Asn
Asn Cys Leu Leu His Pro Met Ser Gln His Gly Ile Glu Asp Pro Glu
Lys Glu Val Leu Glu Trp Arg Phe Asp Ser Lys Leu Ala Phe His His
Val Ala Arg Glu Leu His Pro Glu Tyr Tyr Lys Asp Cys.

Therefore, this exemplified Nef protein, Opt tPA-Nef, contains both a tPA leader sequence as well as deleting the myristylation site of Gly-2A DNA molecule encoding HIV-1 Nef from the HIV-1 jfrl isolate wherein the codons are optimized for expression in a mammalian system such as a human.

In another specific embodiment of the present invention, a DNA molecule is disclosed which encodes optimized HIV-1 Nef wherein the open reading frame codes for modifications at the amino terminal myristylation site (Gly-2 to Ala-2) and substitution of the Leu-174-Leu-175 dileucine motif to Ala-174-Ala-175. This open reading frame is herein described as opt nef (G2A,LLAA) and is disclosed as SEQ ID NO:5, which comprises an initiating methionine residue at nucleotides 12-14 and a “TAA” stop codon from nucleotides 660-662. The nucleotide sequence of this codon optimized version of HIV-1 jrfl nef gene with the above mentioned modifications is disclosed herein as SEQ ID NO:5, as follows:

(SEQ ID NO:5)
GATCTGCCAC CATGGCCGGC AAGTGGTCCA AGAGGTCCGT GCCCGGCTCG TCCACCGTCA
GGGAGAGGAT GAGGAGGGCC GAGCCCGCCG CCGACAGGGT GAGGAGGACC GAGCCCGCCG
CCGTGGGCGT GGGCGCCGTG TCCAGGGACC TGGAGAAGCA CGGCGCCATC ACCTCCTCCA
ACACCGCCGC CACCAACGCC GACTGCGCCT GGCTGGAGGC CCAGGAGGAC GAGGAGGTGG
GCTTCCCCGT GAGGCCCCAG GTGCCCCTGA GGCCCATGAC CTACAAGGGC GCCGTGGACC
TGTCCCACTT CCTGAAGGAG AAGGGCGGCC TGGAGGGCCT GATCCACTCC CAGAAGAGGC
AGGACATCCT GGACCTGTGG GTGTACCACA CCCAGGGCTA CTTCCCCGAC TGGCAGAACT
ACACCCCCGG CCCCGGCATC AGGTTCCCCC TGACCTTCGG CTGGTGCTTC AAGCTGGTGC
CCGTGGAGCC CGAGAAGGTG GAGGAGGCCA ACGAGGGCGA GAACAACTGC GCCGCCCACC
CCATGTCCCA GCACGGCATC GACCACCCCG AGAAGGAGGT GCTGGAGTGG AGGTTCCACT
CCAAGCTGGC CTTCCACCAC GTGGCCAGGG AGCTGCACCC CGAGTACTAC AAGGACTGCT
AAAGCCCGGG C.

The open reading frame of SEQ ID NO:5 encodes Nef (G2A,LLAA), disclosed herein as SEQ ID NO:6, as follows:

(SEQ ID NO:6)
Met Ala Gly Lys Trp Ser Lys Arg Ser Val Pro Gly Trp Ser Thr Val
Arg Glu Arg Met Arg Arg Ala Glu Pro Ala Ala Asp Arg Val Arg Arg
Thr Glu Pro Ala Ala Val Gly Val Gly Ala Val Ser Arg Asp Leu Glu
Lys His Gly Ala Ile Thr Ser Ser Asn Thr Ala Ala Thr Asn Ala Asp
Cys Ala Trp Leu Glu Ala Cln Glu Asp Glu Glu Val Gly Phe Pro Val
Arg Pro Gln Val Pro Leu Arg Pro Met Thr Tyr Lys Gly Ala Val Asp
Leu Ser His Phe Leu Lys Glu Lys Gly Gly Leu Glu Gly Leu Ile His
Ser Gln LYS Arg Gln Asp Ile Leu Asp Leu Trp Val Tyr His Thr Gln
Gly Tyr Phe Pro Asp Trp Gln Asn Tyr Thr Pro Gly Pro Gly Ile Arg
Phe Pro Leu Thr Phe Gly Trp Cys Phe Lys Leu Val Pro Val Glu Pro
Glu Lys Val Glu Glu Ala Asn Glu Gly Glu Asn Asn Cys Ala Ala His
Pro Met Ser Gln His Gly Ile Glu Asp Pro Glu Lys Glu Val Leu Glu
Trp Arg Phe Asp Ser Lys Leu Ala Phe His His Val Ala Arg Glu Leu
His Pro Glu Tyr Tyr Lys Asp Cys Ser.

An additional embodiment of the present invention relates to another DNA molecule encoding optimized HIV-1 Nef wherein the amino terminal myristylation site and dileucine motif have been deleted, as well as comprising a tPA leader peptide. This DNA molecule, opt tpanef (LLAA) comprises an open reading frame which encodes a Nef protein containing a tPA leader sequence fused to amino acid residue 6-216 of HIV-1 Nef(jfrl), wherein Leu-174 and Leu-175 are substituted with Ala-174 and Ala-175 (Ala-195 and Ala-196 in this tPA-based fusion protein). The nucleotide sequence comprising the open reading frame of opt tpanef (LLAA) is disclosed herein as SEQ ID NO:7, as shown below:

(SEQ ID NO:7)
CATGGATGCA ATGAAGAGAG GGCTCTGCTG TGTGCTGCTG CTGTGTGGAG CAGTCTTCGT
TTCGCCCAGC GAGATCTCCT CCAAGAGGTC CGTGCCCGGC TGGTCCACCG TGAGCGAGAG
GATGAGGAGC GCCGAGCCCG CCGCCGACAG GGTGAGGAGG ACCGAGCCCG CCGCCGTGGG
CGTGGGCGCC GTGTCCAGGG ACCTGGAGAA GCACGCCGCC ATCACCTCCT CCAACACCGC
CGCCACCAAC GCCGACTGCG CCTGGCTGGA GGCCCAGGAG GACGAGGAGG TGGGCTTCCC
CGTGAGGCCC CAGGTGCCCC TGAGGCCCAT GACCTACAAG GGCGCCGTGC ACCTGTCCCA
CTTCCTGAAG GACAAGGGCG GCCTCGAGGG CCTGATCCAC TCCCAGAAGA GGCAGGACAT
CCTGGACCTG TGGGTGTACC ACACCCAGGG CTACTTCCCC GACTGGCAGA ACTACACCCC
CGGCCCCCGC ATCAGOTTCC CCCTGACCTT CGGCTGGTGC TTCAAGCTGG TGCCCGTGGA
GCCCGAGAAG GTGGAGGAGG CCAACGAGGG CGAGAACAAC TGCGCCGCCC ACCCCATGTC
CCAGCACGGC ATCGAGGACC CCGAGAAGGA GGTGCTGGAG TGGAGGTTCG ACTCCAAGCT
GGCCTTCCAC CACGTCGCCA GGGAGCTGCA CCCCGACTAC TACAAGGACT GCTAAAGCCC.

The open reading frame of SEQ ID NO:7 encoding tPA-Nef (LLAA), disclosed herein as SEQ ID NO:8, is as follows:

(SEQ ID NO:8)
Met Asp Ala Met Lys Arg Gly Leu Cys Cys Val Leu Leu Leu Cys Gly
Ala Val Phe Val Ser Pro Ser Glu Ile Ser Ser Lys Arg Ser Val Pro
Gly Trp Ser Thr Val Arg Glu Arg Met Arg Arg Ala Glu Pro Ala Ala
Asp Arg Val Arg Arg Thr Glu Pro Ala Ala Val Gly Val Gly Ala Val
Ser Arg Asp Leu Glu Lys His Gly Ala Ile Thr Ser Ser Asn Thr Ala
Ala Thr Asn Ala Asp Cys Ala Trp Leu Glu Ala Gln Glu Asp Glu Glu
Val Gly Phe Pro Val Arg Pro Gln Val Pro Leu Arg Pro Met Thr Tyr
Lys Gly Ala Val Asp Leu Ser His Phe Leu Lys Glu Lys Gly Gly Leu
Glu Gly Leu Ile His Ser Gln Lys Arg Gln Asp Ile Leu Asp Leu Trp
Val Tyr His Thr Gln Gly Tyr Phe Pro Asp Trp Gln Asn Tyr Thr Pro
Gly Pro Gly Ile Arg Phe Pro Leu Thr Phe Gly Trp Cys Phe Lys Leu
Val Pro Val Glu Pro Glu Lys Val Glu Glu Ala Asn Glu Gly Glu Asn
Asn Cys Ala Ala His Pro Met Ser Gln His Gly Ile Glu Asp Pro Glu
Lys Glu Val Leu Glu Trp Arg Phe Asp Ser Lys Leu Ala Phe His His
Val Ala Arg Glu Leu His Pro Glu Tyr Tyr Lys Asp Cys.

The present invention also relates in part to any DNA molecule, regardless of codon usage, which expresses a wild type or modified Nef protein as described herein, including but not limited to modified Nef proteins which comprise a deletion or substitution of Gly 2, a deletion of substitution of Leu 174 and Leu 175 and/or inclusion of a leader sequence. Therefore, partial or fully codon optimized DNA vaccine expression vector constructs are preferred since such constructs should result in increased host expression. However, it is within the scope of the present invention to utilize “non-codon optimized” versions of the constructs disclosed herein, especially modified versions of HIV Nef which are shown to promote a substantial cellular immune response subsequent to host administration.

The DNA backbone of the DNA vaccines of the present invention are preferably DNA plasmid expression vectors. DNA plasmid expression vectors are well known in the art and the present DNA vector vaccines may be comprised of any such expression backbone which contains at least a promoter for RNA polymerase transcription, and a transcriptional terminator 3′ to the HIV nef coding sequence. In one preferred embodiment, the promoter is the Rous sarcoma virus (RSV) long terminal repeat (LTR) which is a strong transcriptional promoter. A more preferred promoter is the cytomegalovirus promoter with the intron A sequence (CMV-intA). A preferred transcriptional terminator is the bovine growth hormone terminator. In addition, to assist in large scale preparation of an HIV nef DNA vector vaccine, an antibiotic resistance marker is also preferably included in the expression vector. Ampicillin resistance genes, neomycin resistance genes or any other pharmaceutically acceptable antibiotic resistance marker may be used. In a preferred embodiment of this invention, the antibiotic resistance gene encodes a gene product for neomycin resistance. Further, to aid in the high level production of the pharmaceutical by fermentation in prokaryotic organisms, it is advantageous for the vector to contain an origin of replication and be of high copy number. Any of a number of commercially available prokaryotic cloning vectors provide these benefits. In a preferred embodiment of this invention, these functionalities are provided by the commercially available vectors known as pUC. It is desirable to remove non-essential DNA sequences. Thus, the lacZ and lacI coding sequences of pUC are removed in one embodiment of the invention.

DNA expression vectors exemplified herein are also disclosed in PCT International Application No. PCT/US94/02751, International Publication No. WO 94/21797, hereby incorporated by reference. A first DNA expression vector is the expression vector pnRSV, wherein the rous sarcoma virus (RSV) long terminal repeat (LTR) is used as the promoter. A second embodiment relates to plasmid V1, a mutated pBR322 vector into which the CMV promoter and the BGH transcriptional terminator is cloned. Another embodiment regarding DNA vector backbones relates to plasmid V1J. Plasmid V1J is derived from plasmid V1 and removes promoter and transcription termination elements in order to place them within a more defined context, create a more compact vector, and to improve plasmid purification yields. Therefore, V1J also contains the CMVintA promoter and (BGH) transcription termination elements which control the expression of the HIV nef-based genes disclosed herein. The backbone of V1J is provided by pUC18. It is known to produce high yields of plasmid, is well-characterized by sequence and function, and is of minimum size. The entire lac operon was removed and the remaining plasmid was purified from an agarose electrophoresis gel, blunt-ended with the T4 DNA polymerase, treated with calf intestinal alkaline phosphatase, and ligated to the CMVintA/BGH element. In another DNA expression vector, the ampicillin resistance gene is removed from V1J and replaced with a neomycin resistance gene, to generate V1Jneo. A DNA expression vector specifically exemplified herein is V1Jns, which is the same as V1J except that a unique Sfi1 restriction site has been engineered into the single Kpn1 site at position 2114 of V1J-neo. The incidence of Sfi1 sites in human genomic DNA is very low (approximately 1 site per 100,000 bases). Thus, this vector allows careful monitoring for expression vector integration into host DNA, simply by Sfi1 digestion of extracted genomic DNA. Another DNA expression vector for use as the backbone to the HIV-1 nef-based DNA vaccines of the present invention is V1R. In this vector, as much non-essential DNA as possible is “trimmed” from the vector to produce a highly compact vector. This vector is a derivative of V1Jns. This vector allows larger inserts to be used, with less concern that undesirable sequences are encoded and optimizes uptake by cells when the construct encoding specific influenza virus genes is introduced into surrounding tissue.

It will be evident upon review of the teaching within this specification that numerous vector/Nef antigen constructs may be generated. While the exemplified constructs (V1Jns/nef, V1Jns/tpanef, V1Jns/tpanef(LLAA) and V1Jns/(G2A,LLAA) are preferred, any number of vector/Nef antigen combinations are within the scope of the present invention, especially wild type or modified Nef proteins which comprise a deletion or substitution of Gly 2, a deletion of substitution of Leu 174 and Leu 175 and/or inclusion of a leader sequence. Therefore, the present invention especially relates to DNA vaccines and a pharmaceutically active vaccine composition which contains this DNA vector vaccine, and the use as prophylactic and/or therapeutic vaccine for host immunization, preferably human host immunization, against an HIV infection or to combat an existing HIV condition. These DNA vaccines are represented by codon optimized DNA molecules encoding HIV-1 Nef of biologically active Nef modifications or Nef-containing fusion proteins which are ligated within an appropriate DNA plasmid vector, with or without a nucleotide sequence encoding a functional leader peptide. DNA vaccines of the present invention include but in no way are limited to codon optimized DNA molecules encoding HIV-1 Nef of biologically active Nef modifications or Nef-containing fusion proteins ligated in DNA vectors V1, V1J (SEQ ID NO:14), V1Jneo (SEQ ID NO:15), V1Jns (FIG. 1A, SEQ ID NO:16), V1R (SEQ ID NO:26), or any of the aforementioned vectors wherein a nucleotide sequence encoding a leader peptide, preferably the human tPA leader, is fused directly downstream of the CMV-intA promoter, including but not limited to V1Jns-tpa, as shown in FIG. 1B and SEQ ID NO:19. Especially preferred DNA vaccines of the present invention include as V1Jns/nef, V1Jns/tpanef, V1Jns/tpanef(LLAA) and V1Jns/(G2A,LLAA), as exemplified in Example Section 2.

The DNA vector vaccines of the present invention may be formulated in any pharmaceutically effective formulation for host administration. Any such formulation may be, for example, a saline solution such as phosphate buffered saline (PBS). It will be useful to utilize pharmaceutically acceptable formulations which also provide long-term stability of the DNA vector vaccines of the present invention. During storage as a pharmaceutical entity, DNA plasmid vaccines undergo a physiochemical change in which the supercoiled plasmid converts to the open circular and linear form. A variety of storage conditions (low pH, high temperature, low ionic strength) can accelerate this process. Therefore, the removal and/or chelation of trace metal ions (with succinic or malic acid, or with chelators containing multiple phosphate ligands) from the DNA plasmid solution, from the formulation buffers or from the vials and closures, stabilizes the DNA plasmid from this degradation pathway during storage. In addition, inclusion of non-reducing free radical scavengers, such as ethanol or glycerol, are useful to prevent damage of the DNA plasmid from free radical production that may still occur, even in apparently demetalated solutions. Furthermore, the buffer type, pH, salt concentration, light exposure, as well as the type of sterilization process used to prepare the vials, may be controlled in the formulation to optimize the stability of the DNA vaccine. Therefore, formulations that will provide the highest stability of the DNA vaccine will be one that includes a demetalated solution containing a buffer (phosphate or bicarbonate) with a pH in the range of 7-8, a salt (NaCl, KCl or LiCl) in the range of 100-200 mM, a metal ion chelator (e.g., EDTA, diethylenetriaminepenta-acetic acid (DTPA), malate, inositol hexaphosphate, tripolyphosphate or polyphosphoric acid), a non-reducing free radical scavenger (e.g. ethanol, glycerol, methionine or dimethyl sulfoxide) and the highest appropriate DNA concentration in a sterile glass vial, packaged to protect the highly purified, nuclease free DNA from light. A particularly preferred formulation which will enhance long term stability of the DNA vector vaccines of the present invention would comprise a Tris-HCl buffer at a pH from about 8.0 to about 9.0; ethanol or glycerol at about 3% w/v; EDTA or DTPA in a concentration range up to about 5 mM; and NaCl at a concentration from about 50 mM to about 500 mM. The use of such stabilized DNA vector vaccines and various alternatives to this preferred formulation range is described in detail in PCT International Application No. PCT/US97/06655, PCT International Publication No. WO 97/40839, which is hereby incorporated by reference.

The DNA vector vaccines of the present invention may, in addition to generating a strong CTL-based immune response, provide for a measurable humoral response subsequent immunization. This response may occur with or without the addition of adjuvant to the respective vaccine formulation. To this end, the DNA vector vaccines of the present invention may also be formulated with an adjuvant or adjuvants which may increase immunogenicity of the DNA polynucleotide vaccines of the present invention. A number of these adjuvants are known in the art and are available for use in a DNA vaccine, including but not limited to particle bombardment using DNA-coated gold beads, co-administration of DNA vaccines with plasmid DNA expressing cytokines, chemokines, or costimulatory molecules, formulation of DNA with cationic lipids or with experimental adjuvants such as saponin, monophosphoryl lipid A or other compounds which increase immunogenicity of the DNA vaccine. One preferred adjuvant for use in the DNA vector vaccines of the present invention are one or more forms of an aluminum phosphate-based adjuvant. Aluminum phosphate is known in the art for use with live, killed or subunit vaccines, but is only recently disclosed as a useful adjuvant in DNA vaccine formulations. The artisan may alter the ratio of DNA to aluminum phosphate to provide for an optimal immune response. In addition, the aluminum phosphate-based adjuvant possesses a molar PO₄/A1 ratio of approximately 0.9, and may again be altered by the skilled artisan to provide for an optimal immune response. An additional mineral-based adjuvant may be generated from one or more forms of a calcium phosphate. These mineral-based adjuvants are useful in increasing humoral responses to DNA vaccination without imparting a negative effect on an appropriate cellular immune response. Complete guidance for use of these mineral-based compounds for use as DNA vaccines adjuvants are disclosed in PCT International Application No. PCT/US98/02414, PCT International Publication No. WO 98/35562, which are hereby incorporated by reference in their entirety. Another preferred adjuvant is a non-ionic block copolymer which shows adjuvant activity with DNA vaccines. The basic structure comprises blocks of polyoxyethylene (POE) and polyoxypropylene (POP) such as a POE-POP-POE block copolymer. Newman et al. (1998, Critical Reviews in Therapeutic Drug Carrier Systems 15 (2): 89-142) review a class of non-ionic block copolymers which show adjuvant activity. The basic structure comprises blocks of polyoxyethylene (POE) and polyoxypropylene (POP) such as a POE-POP-POE block copolymer. Newman et al. id., disclose that certain POE-POP-POE block copolymers may be useful as adjuvants to an influenza protein-based vaccine, namely higher molecular weight POE-POP-POE block copolymers containing a central POP block having a molecular weight of over about 9000 daltons to about 20,000 daltons and flanking POE blocks which comprise up to about 20% of the total molecular weight of the copolymer (see also U.S. Reissue Pat. No. 36,665, U.S. Pat. No. 5,567,859, U.S. Pat. No. 5,691,387, U.S. Pat. No. 5,696,298 and U.S. Pat. No. 5,990,241, all issued to Emanuele, et al., regarding these POE-POP-POE block copolymers). WO 96/04932 further discloses higher molecular weight POE/POP block copolymers which have surfactant characteristics and show biological efficacy as vaccine adjuvants. The above cited references within this paragraph are hereby incorporated by reference in their entirety. It is therefore within the purview of the skilled artisan to utilize available adjuvants which may increase the immune response of the polynucleotide vaccines of the present ivention in comparison to administration of a non-adjuvanted polynucleotide vaccine.

The DNA vector vaccines of the present invention are administered to the host by any means known in the art, such as enteral and parenteral routes. These routes of delivery include but are not limited to intramusclar injection, intraperitoneal injection, intravenous injection, inhalation or intranasal delivery, oral delivery, sublingual administration, subcutaneous administration, transdermal administration, transcutaneous administration, percutaneous administration or any form of particle bombardment, such as a biolostic device such as a “gene gun” or by any available needle-free injection device. The preferred methods of delivery of the HIV-1 Nef-based DNA vaccines disclosed herein are intramuscular injection and needle-free injection. An especially preferred method is intramuscular delivery.

The amount of expressible DNA to be introduced to a vaccine recipient will depend on the strength of the transcriptional and translational promoters used in the DNA construct, and on the immunogenicity of the expressed gene product. In general, an immunologically or prophylactically effective dose of about 1 μg to greater than about 20 mg, and preferably in doses from about 1 mg to about 5 mg is administered directly into muscle tissue. As noted above, subcutaneous injection, intradermal introduction, impression through the skin, and other modes of administration such as intraperitoneal, intravenous, inhalation and oral delivery are also contemplated. It is also contemplated that booster vaccinations are to be provided in a fashion which optimizes the overall immune response to the Nef-based DNA vector vaccines of the present invention.

The aforementioned polynucleotides, when directly introduced into a vertebrate in vivo, express the respective HIV-1 Nef protein within the animal and in turn induce a cytotoxic T lymphocyte (CTL) response within the host to the expressed Nef antigen. To this end, the present invention also relates to methods of using the HIV-1 Nef-based polynucleotide vaccines of the present invention to provide effective immunoprophylaxis, to prevent establishment of an HIV-1 infection following exposure to this virus, or as a post-HIV infection therapeutic vaccine to mitigate the acute HIV-1 infection so as to result in the establishment of a lower virus load with beneficial long term consequences. As noted above, the present invention contemplates a method of administration or use of the DNA nef-based vaccines of the present invention using an any of the known routes of introducing polynucleotides into living tissue to induce expression of proteins.

Therefore, the present invention provides for methods of using a DNA nef-based vaccine utilizing the various parameters disclosed herein as well as any additional parameters known in the art, which, upon introduction into mammalian tissue induces in vivo, intracellular expression of these DNA nef-based vaccines. This intracellular expression of the Nef-based immunogen induces a CTL and humoral response which provides a substantial level of protection against an existing HIV-1 infection or provides a substantial level of protection against a future infection in a presently uninfected host.

The following examples are provided to illustrate the present invention without, however, limiting the same hereto.

### EXAMPLE 1

### Vaccine Vectors

V1—Vaccine vector V1 was constructed from pCMVIE-AKI-DHFR (Whang et al., 1987, J. Virol. 61: 1796). The AKI and DHFR genes were removed by cutting the vector with EcoRI and self-ligating. This vector does not contain intron A in the CMV promoter, so it was added as a PCR fragment that had a deleted internal SacI site [at 1855 as numbered in Chapman, et al., (1991, Nuc. Acids Res. 19: 3979)]. The template used for the PCR reactions was pCMVintA-Lux, made by ligating the HindIII and NheI fragment from pCMV6a120 (see Chapman et al., ibid.), which includes hCMV-IE1 enhancer/promoter and intron A, into the HindIII and XbaI sites of pBL3 to generate pCMVIntBL. The 1881 base pair luciferase gene fragment (HindIII-SmaI Klenow filled-in) from RSV-Lux (de Wet et al., 1987, Mol. Cell Biol. 7: 725) was ligated into the SalI site of pCMVIntBL, which was Klenow filled-in and phosphatase treated. The primers that spanned intron A are: 5′ primer: 5′-CTATATAAGCAGAGCTCGTTTAG-3′ (SEQ ID NO:10); 3′ primer: 5′-GTAGCAAAGATCTAAGGACGGTGACTGCAG-3′ (SEQ ID NO:11). The primers used to remove the SacI site are: sense primer, 5′-GTATGTGTCTG AAAATGAGC GTGGAGATTGGGCTCGCAC-3′ (SEQ ID NO:12) and the antisense primer, 5′-GTGCGAGCCCAATCTCCACGCTCATTTTCAGAC ACATAC-3′ (SEQ ID NO:13). The PCR fragment was cut with Sac I and Bgl II and inserted into the vector which had been cut with the same enzymes.

V1J—Vaccine vector V1J was generated to remove the promoter and transcription termination elements from vector V1 in order to place them within a more defined context, create a more compact vector, and to improve plasmid purification yields. V1J is derived from vectors V1 and pUC18, a commercially available plasmid. V1 was digested with SspI and EcoRI restriction enzymes producing two fragments of DNA. The smaller of these fragments, containing the CMVintA promoter and Bovine Growth Hormone (BGH) transcription termination elements which control the expression of heterologous genes, was purified from an agarose electrophoresis gel. The ends of this DNA fragment were then “blunted” using the T4 DNA polymerase enzyme in order to facilitate its ligation to another “blunt-ended” DNA fragment. pUC18 was chosen to provide the “backbone” of the expression vector. It is known to produce high yields of plasmid, is well-characterized by sequence and function, and is of small size. The entire lac operon was removed from this vector by partial digestion with the HaeII restriction enzyme. The remaining plasmid was purified from an agarose electrophoresis gel, blunt-ended with the T4 DNA polymerase treated with calf intestinal alkaline phosphatase, and ligated to the CMVintA/BGH element described above. Plasmids exhibiting either of two possible orientations of the promoter elements within the pUC backbone were obtained. One of these plasmids gave much higher yields of DNA in E. coli and was designated V1J. This vector's structure was verified by sequence analysis of the junction regions and was subsequently demonstrated to give comparable or higher expression of heterologous genes compared with V1. The nucleotide sequence of V1J is as follows:

(SEQ ID NO:14)
TCGCGCGTTT CGGTGATGAC GGTGAAAACC TCTGACACAT GCAGCTCCCG GAGACGGTCA
CAGCTTGTCT GTAAGCGGAT GCCGGGAGCA GACAAGCCCG TCAGCGCGCG TCAGCGGGTG
TTGGCGGGTG TCGCGGCTGG CTTAACTATG CGGCATCAGA GCAGATTGTA CTGAGAGTGC
ACCATATGCG GTGTGAAATA CCGCACACAT GCGTAAGGAG AAAATACCGC ATCAGATTGG
CTATTGGCCA TTGCATACGT TGTATCCATA TCATAATATG TACATTTATA TTGGCTCATG
TCCAACATTA CCGCCATGTT GACATTGATT ATTGACTAGT TATTAATAGT AATCAATTAC
GGGGTCATTA GTTCATAGCC CATATATGGA GTTCCGCGTT ACATAACTTA CGGTAAATGG
CCCGCCTGGC TGACCGCCCA ACGACCCCCG CCCATTGACG TCAATAATGA CGTATGTTCC
CATAGTAACG CCAATAGGGA CTTTCCATTC ACGTCAATGG GTGGAGTATT TACGGTAAAC
TGCCCACTTG GCAGTACATC AAGTGTATCA TATGCCAAGT ACGCCCCCTA TTGACGTCAA
TGACGGTAAA TGGCCCGCCT GGCATTATGC CCAGTACATG ACCTTATGGG ACTTTCCTAC
TTGGCAGTAC ATCTACGTAT TAGTCATCGC TATTACCATG GTGATGCGGT TTTGGCAGTA
CATCAATGGG CGTGGATAGC GGTTTGACTC ACGGGGATTT CCAAGTCTCC ACCCCATTGA
CGTCAATGGG AGTTTGTTTT GGCACCAAAA TCAACGGGAC TTTCCAAAAT GTCCTAACAA
CTCCGCCCCA TTGACGCAAA TGGGCGGTAG GCGTGTACGG TGGGAGGTCT ATATAAGCAG
AGCTCGTTTA GTGAACCGTC AGATCGCCTG GAGACGCCAT CCACGCTGTT TTGACCTCCA
TACAAGACAC CGGGACCGAT CCAGCCTCCG CGGCCGGGAA CCGTGCATTG GAACGCGGAT
TCCCCGTGCC AAGAGTGACG TAAGTACCGC CTATAGAGTC TATAGGCCCA CCCCCTTGGC
TTCTTATGCA TGCTATACTG TTTTTGGCTT GGGGTCTATA CACCCCCGCT TCCTCATGTT
ATAGGTGATG GTATAGCTTA GCCTATAGGT GTGGGTTATT GACCATTAAT GACCACTCCC
CTATTGGTGA CGATACTTTC CATTACTAAT CCATAACATG GCTCTTTGCC ACAACTCTCT
TTATTGGCTA TATGCCAATA CACTGTCCTT CAGAGACTGA CACGGACTCT GTATTTTTAC
AGGATGGGGT CTCATTTATT ATTTACAAAT TCACATATAC AACACCACCG TCCCCAGTGC
CCGCAGTTTT TATTAAACAT AACGTGGGAT CTCCACGCGA ATCTCGGGTA CGTGTTCCGG
ACATGGGCTC TTCTCCGGTA GCGGCGGAGC TTCTACATCC GAGCCCTGCT CCCATGCCTC
CAGCGACTCA TCGTCGCTCG GCAGCTCCTT GCTCCTAACA GTGGAGGCCA GACAAACGCA
CAGCACGATG CCCACCACCA CCAGTGTGCC GCACAAGGCC GTGGCGGTAC GGTATGTGTC
TGAAAATGAG CTCGGGGAGC GGGCTTGCAC CGCTGACGCA TTTGGAAGAC TTAAGGCAGC
GGCAGAAGAA GATGCAGGCA GCTGAGTTGT TGTGTTCTGA TAAGAGTCAG AGGTAACTCC
CGTTGCGGTG CTGTTAACGG TGGAGGGCAG TGTAGTCTGA CCACTACTCG TTGCTGCCGC
GCGCGCCACC AGACATAATA GCTGACAGAC TAACAGACTG TTCCTTTCCA TGGGTCTTTT
CTGCACTCAC CGTCCTTAGA TCTGCTGTGC CTTCTAGTTG CCAGCCATCT GTTGTTTGCC
CCTCCCCCGT GCCTTCCTTG ACCCTGGAAG GTGCCACTCC CACTGTCCTT TCCTAATAAA
ATGAGGAAAT TCCATCGCAT TGTCTGAGTA GCTGTCATTC TATTCTGGGG GGTGGGGTGG
GGCAGCACAG CAAGGGGGAG GATTGGCAAG ACAATAGCAG GCATGCTGGG GATGCGGTGG
GCTCTATGGG TACCCAGGTG CTGAAGAATT GACCCGGTTC CTCCTGGGCC AGAAAGAAGC
AGGCACATCC CCTTCTCTGT GACACACCCT CTCCACGCCC CTCGTTCTTA GTTCCAGCCC
CACTCATAGG ACACTCATAG CTCAGGAGGG CTCCGCCTTC AATCCCACCC GCTAAAGTAC
TTGGAGCGGT CTCTCCCTCC CTCATCAGCC CACCAAACCA AACCTAGCCT CCAAGAGTGG
GAAGAAATTA AAGCAAGATA GGCTATTAAG TGCAGAGGGA GAGAAAATGC CTCCAACATG
TGACGAAGTA ATGAGAGAAA TCATAGAATT TCTTCCCCTT CCTCGCTCAC TGACTCGCTG
CGCTCGGTCG TTCGGCTGCG GCGAGCGGTA TCAGCTCACT CAAAGGCGGT AATACGGTTA
TCCACAGAAT CAGGGGATAA CGCAGGAAAG AACATGTGAG CAAAAGGCCA GCAAAAGGCC
AGGAACCGTA AAAAGCCCGC GTTCCTGGCG TTTTTCCATA GGCTCCOCCC CCCTGACGAG
CATCACAAAA ATCGACGCTC AAGTCAGAGG TGGCGAAACC CGACAGGACT ATAAAGATAC
CAGOCGTTTC CCCCTGGAAG CTCCCTCGTG CGCTCTCCTG TTCCGACCCT GCCGCTTACC
GGATACCTGT CCGCCTTTCT CCCTTCGGGA AGCGTGGCGC TTTCTCAATG CTCACGCTGT
AGGTATCTCA GTTCGGTGTA GGTCGTTCGC TCCAAGCTGG CCTGTGTGCA CGAACCCCCC
GTTCAGCCCG ACCGCTGCGC CTTATCCCCT AACTATCGTC TTGAGTCCAA CCCGGTAAGA
CACGACTTAT CGCCACTGGC AGCAGCCACT GGTAACAGGA TTAGCAGAGC GAGGTATGTA
GGCGGTGCTA CAGAGTTCTT GAAGTGGTGG CCTAACTACG GCTACACTAG AAGGACAGTA
TTTGGTATCT GCGCTCTGCT GAAGCCAGTT ACCTTCGGAA AAAGAGTTGG TAGCTCTTGA
TCCGGCAAAC AAACCACCGC TGGTAGCGGT GGTTTTTTTG TTTGCAAGCA GCAGATTACG
CGCAGAAAAA AAGGATCTCA AGAAGATCCT TTGATCTTTT CTACGGGGTC TGACGCTCAG
TGGAACGAAA ACTCACGTTA AGGGATTTTG GTCATGAGAT TATCAAAAAG GATCTTCACC
TAGATCCTTT TAAATTAAAA ATGAAGTTTT AAATCAATCT AAAGTATATA TGAGTAAACT
TGGTCTGACA GTTACCAATG CTTAATCAGT GAGGCACCTA TCTCAGCGAT CTGTCTATTT
CGTTCATCCA TAGTTGCCTG ACTCCCCGTC GTGTAGATAA CTACGATACG GGAGGGCTTA
CCATCTGGCC CCAGTGCTGC AATGATACCG CGAGACCCAC GCTCACCGGC TCCAGATTTA
TCAGCAATAA ACCAGCCAGC CGGAAGGGCC GAGCGCAGAA GTGGTCCTGC AACTTTATCC
GCCTCCATCC AGTCTATTAA TTGTTGCCGG GAAGCTAGAG TAAGTAGTTC CCCAGTTAAT
AGTTTGCGCA ACGTTGTTGC CATTOCTACA GGCATCGTGG TGTCACGCTC GTCGTTTGGT
ATGGCTTCAT TCAGCTCCGG TTCCCAACGA TCAAGGCGAG TTACATGATC CCCCATGTTG
TGCAAAAAAG CGGTTAGCTC CTTCGGTCCT CCGATCCTTG TCAGAAGTAA GTTGGCCGCA
GTGTTATCAC TCATGGTTAT GGCAGCACTG CATAATTCTC TTACTGTCAT GCCATCCGTA
AGATGCTTTT CTGTGACTGG TGAGTACTCA ACCAAGTCAT TCTGAGAATA GTGTATGCGG
CGACCGAGTT GCTCTTGCCC CGCGTCAATA CGGGATAATA CCGCGCCACA TAGCAGAACT
TTAAAAGTGC TCATCATTGG AAAACGITCT TCGGGGCGAA AACTCTCAAG GATCTTACCG
CTGTTGAGAT CCAGTTCGAT GTAACCCACT CGTGCACCCA ACTGATCTTC AGCATCTTTT
ACTTTCACCA GCGTTTCTGG GTGAGCAAAA ACAGGAAGGC AAAATGCCGC AAAAAAGGGA
ATAAGGGCGA CACGGAAATG TTGAATACTC ATACTCTTCC TTTTTCAATA TTATTGAAGC
ATTTATCAGG GTTATTGTCT CATGAGCGGA TACATATTTG AATGTATTTA GAAAAATAAA
CAAATAGGGG TTCCGCGCAC ATTTCCCCGA AAAGTGCCAC CTGACGTCTA AGAAACCATT
ATTATCATGA CATTAACCTA TAAAAATAGG CGTATCACGA GGCCCTTTCG TC.

V1Jneo—Construction of vaccine vector V1Jneo expression vector involved removal of the amp^{r} gene and insertion of the kan^{r} gene (neomycin phosphotransferase). The amp^{r} gene from the pUC backbone of V1J was removed by digestion with SspI and Eam1105I restriction enzymes. The remaining plasmid was purified by agarose gel electrophoresis, blunt-ended with T4 DNA polymerase, and then treated with calf intestinal alkaline phosphatase. The commercially available kan^{r} gene, derived from transposon 903 and contained within the pUC4K plasmid, was excised using the PstI restriction enzyme, purified by agarose gel electrophoresis, and blunt-ended with T4 DNA polymerase. This fragment was ligated with the V1J backbone and plasmids with the kan^{r} gene in either orientation were derived which were designated as V1Jneo #'s 1 and 3. Each of these plasmids was confirmed by restriction enzyme digestion analysis, DNA sequencing of the junction regions, and was shown to produce similar quantities of plasmid as V1J. Expression of heterologous gene products was also comparable to V1J for these V1Jneo vectors. V1Jneo#3, referred to as V1Jneo hereafter, was selected which contains the kan^{r} gene in the same orientation as the amp^{r} gene in V1J as the expression construct and provides resistance to neomycin, kanamycin and G418. The nucleotide sequence of V1Jneo is as follows:

| | | | | | | |
|---|---|---|---|---|---|---|
| TCGCGCGTTT | CGGTGATCAC | GGTGAAAACC | TCTGACACAT | GCAGCTCCCC | GAGACGGTCA | (SEQ ID NO:15) |
| CAGCTTGTCT | GTAAGCGGAT | GCCGGGAGCA | GACAAGCCCG | TCAGGCCGCG | TCAGCGGGTG | |
| TTGGCGGGTG | TCGGGGCTGG | CTTAACTATG | CGGCATCAGA | GCAGATTGTA | CTGAGAGTGC | |
| ACCATATGCG | GTGTGAAATA | CCGCACAGAT | GCGTAAGGAG | AAAATACCGC | ATCAGATTGG | |
| CTATTGGCCA | TTGCATACGT | TGTATCCATA | TCATAATATG | TACATTTATA | TTGGCTCATG | |
| TCCAACATTA | CCGCCATGTT | GACATTGATT | ATTGACTAGT | TATTAATAGT | AATCAATTAC | |
| GGGGTCATTA | GTTCATAGCC | CATATATGGA | GTTCCGCGTT | ACATAACTTA | CGGTAAATGG | |
| CCCGCCTCGC | TGACCGCCCA | ACGACCCCCG | CCCATTGACG | TCAATAATGA | CGTATGTTCC | |
| CATAGTAACG | CCAATAGGCA | CTTTCCATTG | ACGTCAATGC | GTGGAGTATT | TACGGTAAAC | |
| TGCCCACTTG | GCAGTACATC | AAGTGTATCA | TATGCCAAGT | ACGCCCCCTA | TTGACGTCAA | |
| TGACGGTAAA | TGGCCCGCCT | GGCATTATCC | CCAGTACATG | ACCTTATGGG | ACTTTCCTAC | |
| TTGGCAGTAC | ATCTACGTAT | TAGTCATCGC | TATTACCATG | GTGATGCGGT | TTTGGCAGTA | |
| CATCAATGGG | CGTGGATAGC | GGTTTGACTC | ACGGGGATTT | CCAAGTCTCC | ACCCCATTGA | |
| CGTCAATGGG | AGTTTGTTTT | GGCACCAAAA | TCAACGGGAC | TTTCCAAAAT | GTCGTAACAA | |
| CTCCGCCCCA | TTGACGCAAA | TGGGCGGTAG | GCGTGTACGG | TGGGAGGTCT | ATATAAGCAG | |
| AGCTCGTTTA | GTGAACCGTC | AGATCGCCTG | GAGACGCCAT | CCACGCTGTT | TTGACCTCCA | |
| TAGAAGACAC | CGGGACCGAT | CCAGCCTCCG | CGGCCGGGAA | CGGTGCATTG | GAACGCGCAT | |
| TCCCCGTGCC | AAGAGTGACG | TAAGTACCGC | CTATAGAGTC | TATAGGCCCA | CCCCCTTGGC | |
| TTCTTATGCA | TGCTATACTG | TTTTTGGCTT | GGGGTCTATA | CACCCCCGCT | TCCTCATGTT | |
| ATAGGTGATG | GTATAGCTTA | GCCTATAGGT | GTCGGTTATT | GACCATTATT | GACCACTCCC | |
| CTATTGGTGA | CGATACTTTC | CATTACTAAT | CCATAACATG | GCTCTTTGCC | ACAACTCTCT | |
| TTATTGGCTA | TATGCCAATA | CACTGTCCTT | CAGAGACTGA | CACGGACTCT | GTATTTTTAC | |
| AGGATGGGGT | CTCATTTATT | ATTTACAAAT | TCACATATAC | AACACCACCG | TCCCCACTGC | |
| CCGCAGTTTT | TATTAAACAT | AACGTGGGAT | CTCCACGCGA | ATCTCGGGTA | CGTGTTCCGG | |
| ACATGGCCTC | TTCTCCGGTA | GCGGCGGAGC | TTCTACATCC | GAGCCCTGCT | CCCATGCCTC | |
| CAGCGACTCA | TGGTCGCTCG | GCAGCTCCTT | GCTCCTAACA | GTGGAGGCCA | GACTTAGGCA | |
| CAGCACGATC | CCCACCACCA | CCAGTGTGCC | GCACAAGGCC | GTGGCGGTAG | GGTATGTGTC | |
| TGAAAATGAG | CTCGGGGAGC | GGGCTTGCAC | CGCTGACGCA | TTTGGAAGAC | TTAAGGCAGC | |
| GGCACAAGAA | GATGCAGGCA | GCTGAGTTGT | TGTGTTCTGA | TAAGAGTCAG | AGGTAACTCC | |
| CGTTGCGGTG | CTGTTAACGG | TGGAGGGCAG | TCTAGTCTGA | GCAGTACTCG | TTGCTGCCGC | |
| GCGCGCCACC | AGACATAATA | GCTGACAGAC | TAACAGACTG | TTCCTTTCCA | TGGGTCTTTT | |
| CTGCAGTCAC | CGTCCTTAGA | TCTGCTGTGC | CTTCTAGTTG | CCAGCCATCT | GTTGTTTGCC | |
| CCTCCCCCGT | GCCTTCCTTG | ACCCTGGAAG | GTGCCACTCC | CACTGTCCTT | TCCTAATAAA | |
| ATGAGGAAAT | TGCATCGCAT | TGTCTGAGTA | GGTGTCATTC | TATTCTGGGG | GCTGGGGTGG | |
| GGCAGCACAG | CAAGGGGGAG | GATTGGGAAG | ACAATAGCAG | GCATGCTGGG | GATGCGGTGG | |
| GCTCTATGGC | TACCCAGGTG | CTGAAGAATT | GACCCGGTTC | CTCCTGGGCC | AGAAAGAAGC | |
| AGGCACATCC | CCTTCTCTGT | GACACACCCT | GTCCACGCCC | CTGGTTCTTA | GTTCCAGCCC | |
| CACTCATAGG | ACACTCATAG | CTCAGGAGGG | CTCCGCCTTC | AATCCCACCC | GCTAAAGTAC | |
| TTGGAGCGGT | CTCTCCCTCC | CTCATCAGCC | CACCAAACCA | AACCTAGCCT | CCAAGAGTGG | |
| GAAGAAATTA | AAGCAAGATA | GGCTATTAAG | TGCAGAGGGA | GAGAAAATGC | CTCCAACATG | |
| TGAGGAAGTA | ATGAGAGAAA | TCATAGAATT | TCTTCCGCTT | CCTCGCTCAC | TGACTCGCTG | |
| CGCTCGGTCG | TTCGGCTGCG | GCGAGCGGTA | TCAGCTCACT | CAAAGGCGGT | AATACGGTTA | |
| TCCACAGAAT | CAGGGGATAA | CGCAGGAAAG | AACATGTGAG | CAAAAGGCCA | GCAAAAGGCC | |
| AGGAACCGTA | AAAAGGCCGC | GTTGCTGGCG | TTTTTCCATA | GGCTCCGCCC | CCCTGACGAG | |
| CATCACAAAA | ATCGACGCTC | AAGTCAGAGG | TGGCGAAACC | CGACAGGACT | ATAAAGATAC | |
| CAGGCGTTTC | CCCCTGGAAG | CTCCCTCGTG | CGCTCTCCTG | TTCCGACCCT | GCCGCTTACC | |
| GGATACCTGT | CCGCCTTTCT | CCCTTCGGGA | AGCGTGGCGC | TTTCTCAATG | CTCACGCTGT | |
| AGGTATCTCA | GTTCGGTGTA | GGTCGTTCGC | TCCAAGCTGG | GCTGTGTGCA | CGAACCCCCC | |
| GTTCAGCCCG | ACCGCTGCGC | CTTATCCGGT | AACTATCGTC | TTGAGTCCAA | CCCGGTAAGA | |
| CACGACTTAT | CGCCACTGGC | AGCAGCCACT | GGTAACAGGA | TTAGCAGAGC | GAGGTATGTA | |
| GGCGGTGCTA | CAGAGTTCTT | GAAGTGGTGG | CCTAACTACG | GCTACACTAG | AAGGACAGTA | |
| TTTGGTATCT | GCGCTCTGCT | GAAGCCAGTT | ACCTTCGGAA | AAAGAGTTGG | TAGCTCTTGA | |
| TCCGGCAAAC | AAACCACCGC | TGGTAGCGGT | GGTTTTTTTG | TTTGCAAGCA | GCAGATTACG | |
| CGCAGAAAAA | AAGGATCTCA | AGAAGATCCT | TTGATCTTTT | CTACGGGGTC | TGACGCTCAG | |
| TGGAACGAAA | ACTCACGTTA | AGGGATTTTG | GTCATGAGAT | TATCAAAAAG | GATCTTCACC | |
| TAGATCCTTT | TAAATTAAAA | ATGAAGTTTT | AAATCAATCT | AAAGTATATA | TGAGTAAACT | |
| TGGTCTGACA | GTTACCAATG | CTTAATCAGT | GAGGCACCTA | TCTCAGCGAT | CTGTCTATTT | |
| CGTTCATCCA | TAGTTGCCTG | ACTCCGGGGG | GGGGGGGCGC | TCAGGTCTCC | CTCGTGAAGA | |
| AGGTGTTGCT | GACTCATACC | AGGCCTGAAT | CGCCCCATCA | TCCAGCCAGA | AAGTGAGGGA | |
| GCCACGGTTG | ATGAGAGCTT | TGTTGTAGGT | GGACCAGTTG | GTGATTTTGA | ACTTTTGCTT | |
| TGCCACGGAA | CGGTCTGCGT | TGTCGGGAAG | ATGCGTGATC | TGATCCTTCA | ACTCAGCAAA | |
| AGTTCGATTT | ATTCAACAAA | GCCGCCGTCC | CGTCAAGTCA | GCGTAATGCT | CTGCCAGTGT | |
| TACAACCAAT | TAACCAATTC | TGATTAGAAA | AACTCATCGA | GCATCAAATG | AAACTGCAAT | |
| TTATTCATAT | CAGGATTATC | AATACCATAT | TTTTGAAAAA | GCCGTTTCTG | TAATGAAGGA | |
| GAAAACTCAC | CGAGGCAGTT | CCATAGGATG | GCAAGATCCT | GGTATCGGTC | TGCGATTCCG | |
| ACTCGTCCAA | CATCAATACA | ACCTATTAAT | TTCCCCTCGT | CAAAAATAAG | GTTATCAAGT | |
| GAGAAATCAC | CATGAGTGAC | GACTGAATCC | GGTGAGAATG | GCAAAAGCTT | ATCCATTTCT | |
| TTCCAGACTT | GTTCAACAGG | CCAGCCATTA | CGCTCGTCAT | CAAAATCACT | CGCATCAACC | |
| AAACCCTTAT | TCATTCGTGA | TTGCGCCTGA | GCGAGACGAA | ATACGCGATC | GCTGTTAAAA | |
| GGACAATTAC | AAACAGGAAT | CGAATGCAAC | CGGCGCAGGA | ACACTGCCAG | CGCATCAACA | |
| ATATTTTCAC | CTGAATCAGG | ATATTCTTCT | AATACCTGGA | ATGCTGTTTT | CCCGGGGATC | |
| GCAGTGGTCA | GTAACCATGC | ATCATCAGGA | GTACGGATAA | AATGCTTGAT | GGTCGGAAGA | |
| GGCATAAATT | CCGTCAGCCA | GTTTAGTCTG | ACCATCTCAT | CTGTAACATC | ATTGGCAACG | |
| CTACCTTTGC | CATGTTTCAG | AAACAACTCT | GGCGCATCGG | GCTTCCCATA | CAATCGATAC | |
| ATTGTCGCAC | CTGATTGCCC | GACATTATCG | CGAGCCCATT | TATACCCATA | TAAATCAGCA | |
| TCCATGTTGG | AATTTAATCG | CGGCCTCGAG | CAAGACGTTT | CCCGTTGAAT | ATGGCTCATA | |
| ACACCCCTTG | TATTACTGTT | TATGTAAGCA | GACAGTTTTA | TTGTTCATGA | TGATATATTT | |
| TTATCTTGTG | CAATGTAACA | TCAGAGATTT | TGAGACACAA | CGTGGCTTTC | CCCCCCCCCC | |
| CATTATTGAA | GCATTTATCA | GGGTTATTCT | CTCATGAGCG | GATACATATT | TGAATGTATT | |
| TAGAAAAATA | AACAAATAGG | GGTTCCGCGC | ACATTTCCCC | GAAAAGTGCC | ACCTGACGTC | |
| TAAGAAACCA | TTATTATCAT | GACATTAACC | TATAAAAATA | GCCGTATCAC | GAGGCCCTTT | |
| CGTC. | | | | | | |

V1Jns—The expression vector VIJns was generated by adding an SfiI site to V1Jneo to facilitate integration studies. A commercially available 13 base pair SfiI linker (New England BioLabs) was added at the KpnI site within the BGH sequence of the vector. V1Jneo was linearized with KpnI, gel purified, blunted by T4 DNA polymerase, and ligated to the blunt SfiI linker. Clonal isolates were chosen by restriction mapping and verified by sequencing through the linker. The new vector was designated V1Jns. Expression of heterologous genes in V1Jns (with SfiI) was comparable to expression of the same genes in V1Jneo (with KpnI).

The nucleotide sequence of V1Jns is as follows:

| | |
|---|---|
| TCGCGCGTTT CGGTGATGAC GGTGAAAACC TCTGACACAT GCAGCTCCCG GAGACGGTCA | (SEQ ID NO:16) |
| CAGCTTGTCT GTAAGCGGAT GCCGGGAGCA GACAAGCCCG TCAGGGCGCG TCAGCGGGTG | |
| TTGGCGGGTG TCGCGGCTGC CTTAACTATG CGGCATCAGA GCAGATTGTA CTGAGAGTGC | |
| ACCATATGCG GTGTGAAATA CCGCACAGAT GCGTAAGGAG AAAATACCGC ATCAGATTGG | |
| CTATTGGCCA TTGCATACGT TGTATCCATA TCATAATATG TACATTTATA TTGGCTCATG | |
| TCCAACATTA CCGCCATGTT GACATTGATT ATTCACTAGT TATTAATAGT AATCAATTAC | |
| GGGGTCATTA GTTCATAGCC CATATATGGA GTTCCGCGTT ACATAACTTA CGGTAAATGG | |
| CCCGCCTGGC TGACCGCCCA ACGACCCCCG CCCATTGACG TCAATAATGA CGTATGTTCC | |
| CATAGTAACG CCAATAGGGA CTTTCCATTG ACGTCAATGG GTGGAGTATT TACGGTAAAC | |
| TGCCCACTTG GCAGTACATC AAGTGTATCA TATGCCAAGT ACGCCCCCTA TTGACGTCAA | |
| TGACGGTAAA TGGCCCGCCT GGCATTATGC CCAGTACATG ACCTTATGGG ACTTTCCTAC | |
| TTGGCAGTAC ATCTACGTAT TAGTCATCGC TATTACCATG GTGATGCGGT TTTGCCAGTA | |
| CATCAATGGG CGTGGATAGC GGTTTGACTC ACGGCGATTT CCAAGTCTCC ACCCCATTGA | |
| CGTCAATGGG AGTTTGTTTT GGCACCAAAA TCAACGGGAC TTTCCAAAAT GTCGTAACAA | |
| CTCCGCCCCA TTGACGCAAA TGGGCGGTAG GCGTCTACGG TGGGAGGTCT ATATAAGCAG | |
| AGCTCGTTTA GTGAACCGTC AGATCGCCTG GAGACGCCAT CCACGCTGTT TTGACCTCCA | |
| TAGAAGACAC CGGGACCGAT CCAGCCTCCG CGGCCGGGAA CGGTGCATTG GAACGCGGAT | |
| TCCCCGTGCC AAGAGTGACG TAAGTACCGC CTATAGACTC TATAGGCACA CCCCTTTGGC | |
| TCTTATGCAT GCTATACTGT TTTTGGCTTG GGGCCTATAC ACCCCCGCTT CCTTATGCTA | |
| TAGGTGATGG TATAGCTTAG CCTATAGCTG TGGGTTATTG ACCATTATTG ACCACTCCCC | |
| TATTGGTGAC GATACTTTCC ATTACTAATC CATAACATGG CTCTTTGCCA CAACTATCTC | |
| TATTGGCTAT ATGCCAATAC TCTGTCCTTC AGAGACTGAC ACGGACTCTG TATTTTTACA | |
| GGATGCGGTC CCATTTATTA TTTACAAATT CACATATACA ACAACGCCGT CCCCCGTGCC | |
| CGCAGTTTTT ATTAAACATA GCGTGGGATC TCCACGCGAA TCTCGGGTAC GTGTTCCGGA | |
| CATGGGCTCT TCTCCGGTAG CGGCGGAGCT TCCACATCCG AGCCCTGGTC CCATGCCTCC | |
| AGCGGCTCAT GGTCGCTCGG CAGCTCCTTG CTCCTAACAG TGGAGGCCAG ACTTAGGCAC | |
| AGCACAATGC CCACCACCAC CAGTCTGCCG CACAAGGCCC TGGCGGTAGG GTATGTGTCT | |
| GAAAATGAGC GTGGAGATTG GGCTCGCACG GCTGACGCAG ATGGAAGACT TAAGGCAGCG | |
| GCACAAGAAG ATGCAGGCAG CTGAGTTGTT GTATTCTGAT AAGAGTCAGA GGTAACTCCC | |
| GTTCCGGTGC TGTTAACGGT GGAGGGCAGT GTAGTCTGAG CAGTACTCGT TGCTGCCGCG | |
| CGCGCCACCA GACATAATAG CTGACAGACT AACAGACTGT TCCTTTCCAT GGGTCTTTTC | |
| TGCAGTCACC GTCCTTAGAT CTGCTGTGCC TTCTAGTTCC CAGCCATCTG TTCTTTGCCC | |
| CTCCCCCGTG CCTTCCTTGA CCCTGGAAGG TGCCACTCCC ACTGTCCTTT CCTAATAAAA | |
| TGAGGAAATT GCATCGCATT GTCTGAGTAG GTGTCATTCT ATTCTGGGGG GTGGGGTGGG | |
| GCAGCACAGC AAGGGGGAGG ATTGGCAAGA CAATAGCAGG CATGCTGGGG ATGCGGTGGG | |
| CTCTATGGCC GCTGCGGCCA GGTGCTGAAG AATTGACCCG GTTCCTCCTC GGCCAGAAAG | |
| AAGCAGGCAC ATCCCCTTCT CTGTGACACA CCCTGTCCAC GCCCCTGGTT CTTAGTTCCA | |
| GCCCCACTCA TAGGACACTC ATAGCTCAGG AGGGCTCCGC CTTCAATCCC ACCCGCTAAA | |
| GTACTTGGAG CGGTCTCTCC CTCCCTCATC AGCCCACCAA ACCAAACCTA GCCTCCAAGA | |
| GTGGGAAGAA ATTAAAGCAA GATAGGCTAT TAAGTGCAGA GGGAGAGAAA ATGCCTCCAA | |
| CATGTGAGGA AGTAATGAGA GAAATCATAG AATTTCTTCC GCTTCCTCGC TCACTGACTC | |
| GCTGCGCTCG GTCGTTCGGC TGCGGCGAGC GGTATCAGCT CACTCAAAGG CGGTAATACG | |
| GTTATCCACA GAATCAGGGG ATAACGCAGG AAAGAACATG TGAGCAAAAG GCCAGCAAAA | |
| GGCCAGGAAC CGTAAAAAGG CCGCGTTGCT GGCGTTTTTC CATAGGCTCC GCCCCCCTGA | |
| CGAGCATCAC AAAAATCGAC GCTCAAGTCA GACGTGGCGA AACCCGACAC GACTATAAAG | |
| ATACCAGGCG TTTCCCCCTG GAAGCTCCCT CGTGCGCTCT CCTGTTCCGA CCCTGCCGCT | |
| TACCGGATAC CTGTCCGCCT TTCTCCCTTC GGGAAGCGTG GCGCTTTCTC ATAGCTCACG | |
| CTGTAGGTAT CTCAGTTCGG TGTAGGTCGT TCGCTCCAAG CTGGGCTGTG TGCACGAACC | |
| CCCCGTTCAG CCCGACCGCT GCGCCTTATC CGGTAACTAT CGTCTTGAGT CCAACCCGGT | |
| AAGACACGAC TTATCGCCAC TGGCAGCAGC CACTGGTAAC AGGATTAGCA GAGCGAGGTA | |
| TGTAGGCGGT GCTACAGAGT TCTTGAAGTG GTGGCCTAAC TACGGCTACA CTAGAAGAAC | |
| AGTATTTGGT ATCTGCGCTC TGCTGAAGCC AGTTACCTTC GGAAAAAGAG TTGGTAGCTC | |
| TTGATCCGGC AAACAAACCA CCGCTGGTAG CGGTGGTTTT TTTGTTTGCA AGCAGCAGAT | |
| TACGCCCAGA AAAAAAGGAT CTCAAGAAGA TCCTTTGATC TTTTCTACGG GGTCTGACGC | |
| TCAGTGGAAC GAAAACTCAC GTTAAGGGAT TTTGGTCATG AGATTATCAA AAAGCATCTT | |
| CACCTAGATC CTTTTAAATT AAAAATGAAG TTTTAAATCA ATCTAAAGTA TATATGAGTA | |
| AACTTGGTCT GACAGTTACC AATGCTTAAT CAGTGAGGCA CCTATCTCAG CGATCTGTCT | |
| ATTTCGTTCA TCCATAGTTG CCTGACTCGG GGGGGGGGGG CGCTGAGGTC TGCCTCGTGA | |
| AGAAGGTGTT GCTGACTCAT ACCACGCCTG AATCCCCCCA TCATCCAGCC AGAAAGTGAG | |
| GGAGCCACGG TTGATGAGAG CTTTGTTGTA GGTGGACCAG TTGGTGATTT TGAACTTTTG | |
| CTTTGCCACG GAACGGTCTG CCTTGTCGGG AAGATGCGTG ATCTGATCCT TCAACTCAGC | |
| AAAAGTTCGA TTTATTCAAC AAAGCCGCCG TCCCGTCAAG TCAGCGTAAT GCTCTGCCAG | |
| TGTTACAACC AATTAACCAA TTCTGATTAG AAAAACTCAT CGAGCATCAA ATGAAACTGC | |
| AATTTATTCA TATCAGGATT ATCAATACCA TATTTTTGAA AAAGCCGTTT CTGTAATGAA | |
| GGAGAAAACT CACCGAGGCA GTTCCATAGG ATGGCAAGAT CCTGGTATCG GTCTGCGATT | |
| CCGACTCGTC CAACATCAAT ACAACCTATT AATTTCCCCT CGTCAAAAAT AAGGTTATCA | |
| AGTGAGAAAT CACCATGAGT GACGACTGAA TCCGGTGAGA ATGGCAAAAG CTTATGCATT | |
| TCTTTCCAGA CTTGTTCAAC AGGCCAGCCA TTACGCTCGT CATCAAAATC ACTCGCATCA | |
| ACCAAACCGT TATTCATTCG TGATTGCGCC TGAGCGAGAC GAAATACGCG ATCGCTGTTA | |
| AAAGGACAAT TACAAACAGG AATCGAATGC AACCCGCGCA GGAACACTGC CAGCGCATCA | |
| ACAATATTTT CACCTGAATC AGGATATTCT TCTAATACCT GGAATGCTGT TTTCCCGGGG | |
| ATCGCAGTGG TGAGTAACCA TGCATCATCA GGAGTACGGA TAAAATGCTT GATGGTCGGA | |
| AGAGGCATAA ATTCCGTCAG CCAGTTTAGT CTGACCATCT CATCTGTAAC ATCATTGGCA | |
| ACGCTACCTT TGCCATGTTT CAGAAACAAC TCTGGCGCAT CGGGCTTCCC ATACAATCGA | |
| TAGATTGTCG CACCTGATTG CCCGACATTA TCGCGAGCCC ATTTATACCC ATATAAATCA | |
| GCATCCATGT TGGAATTTAA TCGCCGCCTC GAGCAAGACG TTTCCCGTTG AATATGGCTC | |
| ATAACACCCC TTGTATTACT GTTTATGTAA GCAGACAGTT TTATTGTTCA TGATGATATA | |
| TTTTTATCTT GTGCAATGTA ACATCAGAGA TTTTGAGACA CAACGTGGCT TTCCCCCCCC | |
| CCCCATTATT GAAGCATTTA TCAGCGTTAT TGTCTCATGA CCGGATACAT ATTTGAATGT | |
| ATTTAGAAAA ATAAACAAAT AGGCGTTCCG CGCACATTTC CCCGAAAAGT GCCACCTGAC | |
| GTCTAAGAAA CCATTATTAT CATGACATTA ACCTATAAAA ATAGGCGTAT CACGAGGCCC | |
| TTTCGTC. | |

The underlined nucleotides of SEQ ID NO:16 represent the Sfi1 site introduced into the Kpn 1 site of V1Jneo.

V1Jns-tPA—The vaccine vector V1Jns-tPA was constructed in order to fuse an heterologous leader peptide sequence to the nef DNA constructs of the present invention. More specifically, the vaccine vector V1Jns was modified to include the human tissue-specific plasminogen activator (tPA) leader. As an exemplification, but by no means a limitation of generating a nef DNA construct comprising an amino-terminal leader sequence, plasmid V1Jneo was modified to include the human tissue-specific plasminogen activator (tPA) leader. Two synthetic complementary oligomers were annealed and then ligated into V1Jneo which had been BglII digested. The sense and antisense oligomers were 5′ GATCACCATGGATGCAATGAAGAGAG GGCTCTGCTGTGTGCTGCTGCTGTGTGGAGCAGTCTTCGTTTCGCCCAG CGA-3′ (SEQ ID NO:17); and, 5′-GATCTCGCTGGGCGAAACGAAGACTGC TCCACACAGCAGCAGCACACAGCAGAGCCCTCTCTTCATTGCATCCAT GGT-3′ (SEQ ID NO:18). The Kozak sequence is underlined in the sense oligomer. These oligomers have overhanging bases compatible for ligation to BglII-cleaved sequences. After ligation the upstream BglII site is destroyed while the downstream BglII is retained for subsequent ligations. Both the junction sites as well as the entire tPA leader sequence were verified by DNA sequencing. Additionally, in order to conform with V1Jns (=V1Jneo with an SfiI site), an SfiI restriction site was placed at the KpnI site within the BGH terminator region of V1Jneo-tPA by blunting the KpnI site with T4 DNA polymerase followed by ligation with an SfiI linker (catalogue #1138, New England Biolabs), resulting in V1Jns-tPA. This modification was verified by restriction digestion and agarose gel electrophoresis. The V1Jns-tpa vector nucleotide sequence is as follows:

| | |
|---|---|
| TCGCGCGTTT CGGTGATGAC GGTGAAAACC TCTGACACAT GCAGCTCCCG GAGACGGTCA | (SEQ ID NO:9) |
| CAGCTTGTCT GTAAGCGGAT GCCGGGAGCA GACAAGCCCG TCAGGGCGCG TCAGCGGGTG | |
| TTGGCGGGTG TCGGGGCTGG CTTAACTATG CGGCATCAGA GCAGATTGTA CTGAGAGTGC | |
| ACCATATGCG GTGTGAAATA CCGCACAGAT GCGTAAGGAG AAAATACCGC ATCAGATTGG | |
| CTATTGGCCA TTGCATACGT TGTATCCATA TCATAATATG TACATTTATA TTGGCTCATG | |
| TCCAACATTA CCGCCATGTT GACATTGATT ATTGACTAGT TATTAATAGT AATCAATTAC | |
| GGGGTCATTA GTTCATAGCC CATATATGGA GTTCCGCGTT ACATAACTTA CGGTAAATGG | |
| CCCGCCTGGC TGACCGCCCA ACGACCCCCG CCCATTGACG TCAATAATGA CGTATGTTCC | |
| CATAGTAACG CCAATAGGGA CTTTCCATTG ACGTCAATCG GTGGAGTATT TACGGTAAAC | |
| TGCCCACTTG GCAGTACATC AAGTGTATCA TATGCCAAGT ACGCCCCCTA TTGACGTCAA | |
| TGACGGTAAA TGGCCCGCCT GGCATTATGC CCAGTACATG ACCTTATGGG ACTTTCCTAC | |
| TTGGCAGTAC ATCTACGTAT TAGTCATCGC TATTACCATG GTGATGCGGT TTTGGCAGTA | |
| CATCAATGGG CGTGGATAGC GGTTTGACTC ACGGGGATTT CCAAGTCTCC ACCCCATTGA | |
| CGTCAATGGC AGTTTGTTTT GGCACCAAAA TCAACGGGAC TTTCCAAAAT GTCGTAACAA | |
| CTCCGCCCCA TTGACGCAAA TGGGCGGTAG GCGTGTACGC TCGGAGCTCT ATATAAGCAG | |
| AGCTCGTTTA GTGAACCGTC AGATCGCCTG GAGACGCCAT CCACGCTGTT TTGACCTCCA | |
| TAGAAGACAC CGGGACCGAT CCAGCCTCCG CGGCCGGGAA CGGTGCATTG GAACGCGGAT | |
| TCCCCGTGCC AAGAGTGACG TAAGTACCGC CTATAGACTC TATAGGCACA CCCCTTTGGC | |
| TCTTATGCAT GCTATACTGT TTTTGGCTTG GGGCCTATAC ACCCCCGCTT CCTTATGCTA | |
| TAGGTGATGG TATAGCTTAG CCTATAGGTG TGGGTTATTG ACCATTATTG ACCACTCCCC | |
| TATTGGTCAC GATACTTTCC ATTACTAATC CATAACATGG CTCTTTGCCA CAACTATCTC | |
| TATTGGCTAT ATGCCAATAC TCTGTCCTTC AGAGACTGAC ACGGACTCTG TATTTTTACA | |
| GGATGGGGTC CCATTTATTA TTTACAAATT CACATATACA ACAACGCCGT CCCCCGTGCC | |
| CGCAGTTTTT ATTAAACATA GCGTGGGATC TCCACGCGAA TCTCGGGTAC GTGTTCCGGA | |
| CATGGGCTCT TCTCCGGTAG CGGCGGAGCT TCCACATCCG AGCCCTGGTC CCATGCCTCC | |
| AGCGGCTCAT GGTCGCTCGG CAGCTCCTTG CTCCTAACAG TGGAGGCCAG ACTTAGGCAC | |
| AGCACAATGC CCACCACCAC CAGTGTGCCG CACAAGGCCG TGGCGGTAGG GTATGTGTCT | |
| GAAAATGAGC GTGGAGATTG GGCTCGCACG GCTGACGCAG ATGGAAGACT TAAGGCAGCG | |
| GCAGAAGAAG ATGCAGGCAG CTGAGTTGTT GTATTCTGAT AAGAGTCAGA GGTAACTCCC | |
| GTTGCGGTGC TGTTAACGGT GGAGGGCAGT GTAGTCTGAG CAGTACTCGT TGCTGCCGCG | |
| CGCGCCACCA GACATAATAG CTGACAGACT AACAGACTGT TCCTTTCCAT GGGTCTTTTC | |
| TGCAGTCACC GTCCTTAGAT CACCATGGAT GCAATGAAGA GAGGGCTCTG CTGTGTGCTG | |
| CTGCTGTGTG GAGCAGTCTT CGTTTCGCCC AGCGAGATCT GCTGTGCCTT CTAGTTGCCA | |
| GCCATCTGTT GTTTGCCCCT CCCCCGTGCC TTCCTTGACC CTGGAAGGTG CCACTCCCAC | |
| TGTCCTTTCC TAATAAAATG AGGAAATTGC ATCGCATTGT CTGAGTAGGT GTCATTCTAT | |
| TCTGGGGGGT GGGGTGGGGC AGGACAGCAA GGGGGAGGAT TGGCAAGACA ATAGCAGGCA | |
| TGCTGGGGAT GCCGTGGGCT CTATGGCCGC TGCGGCCAGG TCCTCAAGAA TTGACCCGGT | |
| TCCTCCTGGG CCAGAAAGAA GCAGGCACAT CCCCTTCTCT GTGACACACC CTGTCCACGC | |
| CCCTGGTTCT TAGTTCCAGC CCCACTCATA GGACACTCAT AGCTCAGGAG GGCTCCGCCT | |
| TCAATCCCAC CCGCTAAAGT ACTTGGAGCG GTCTCTCCCT CCCTCATCAG CCCACCAAAC | |
| CAAACCTAGC CTCCAAGAGT GGGAAGAAAT TAAAGCAAGA TAGGCTATTA AGTGCAGAGG | |
| GAGAGAAAAT GCCTCCAACA TGTGAGGAAG TAATGAGAGA AATCATAGAA TTTCTTCCGC | |
| TTCCTCGCTC ACTGACTCGC TGCGCTCGGT CGTTCGGCTG CGGCGAGCGG TATCAGCTCA | |
| CTCAAAGGCG GTAATACGGT TATCCACAGA ATCAGGGGAT AACGCAGGAA AGAACATGTG | |
| AGCAAAAGGC CAGCAAAAGG CCAGGAACCG TAAAAAGGCC GCGTTGCTGG CGTTTTTCCA | |
| TAGGCTCCGC CCCCCTGACG AGCATCACAA AAATCGACGC TCAAGTCAGA GGTGGCGAAA | |
| CCCGACAGGA CTATAAACAT ACCAGGCGTT TCCCCCTGGA AGCTCCCTCG TGCGCTCTCC | |
| TGTTCCGACC CTGCCGCTTA CCGGATACCT GTCCGCCTTT CTCCCTTCGG GAAGCGTGGC | |
| GCTTTCTCAT AGCTCACGCT GTAGGTATCT CAGTTCGGTG TAGGTCGTTC GCTCCAAGCT | |
| GGGCTGTGTG CACGAACCCC CCGTTCAGCC CGACCGCTGC GCCTTATCCG GTAACTATCG | |
| TCTTGAGTCC AACCCGGTAA GACACGACTT ATCGCCACTG GCAGCAGCCA CTGGTAACAG | |
| GATTAGCAGA GCGAGGTATG TAGGCGGTGC TACAGAGTTC TTGAAGTGGT GGCCTAACTA | |
| CGGCTACACT AGAAGAACAG TATTTGGTAT CTGCGCTCTG CTGAAGCCAG TTACCTTCGG | |
| AAAAAGAGTT GGTAGCTCTT GATCCGGCAA ACAAACCACC GCTGGTAGCG GTGGTTTTTT | |
| TGTTTGCAAG CAGCAGATTA CGCGCAGAAA AAAAGGATCT CAAGAAGATC CTTTGATCTT | |
| TTCTACGGGG TCTGACGCTC AGTGGAACGA AAACTCACGT TAAGGCATTT TGGTCATGAG | |
| ATTATCAAAA AGGATCTTCA CCTAGATCCT TTTAAATTAA AAATGAAGTT TTAAATCAAT | |
| CTAAAGTATA TATGAGTAAA CTTGGTCTGA CAGTTACCAA TGCTTAATCA GTGAGGCACC | |
| TATCTCAGCG ATCTGTCTAT TTCGTTCATC CATAGTTGCC TGACTCGGGG GGGGGGGGCG | |
| CTGAGGTCTG CCTCGTGAAG AAGGTGTTGC TGACTCATAC CAGGCCTGAA TCGCCCCATC | |
| ATCCAGCCAG AAAGTGAGGG AGCCACGGTT GATGAGAGCT TTGTTGTAGG TGGACCAGTT | |
| GGTGATTTTG AACTTTTGCT TTGCCACGGA ACGGTCTGCG TTGTCGGGAA GATGCGTGAT | |
| CTGATCCTTC AACTCAGCAA AAGTTCGATT TATTCAACAA AGCCCCCGTC CCGTCAAGTC | |
| AGCGTAATGC TCTGCCAGTG TTACAACCAA TTAACCAATT CTGATTAGAA AAACTCATCG | |
| AGCATCAAAT GAAACTGCAA TTTATTCATA TCAGGATTAT CAATACCATA TTTTTGAAAA | |
| AGCCGTTTCT GTAATGAAGG ACAAAACTCA CCGAGGCAGT TCCATAGGAT GGCAAGATCC | |
| TGGTATCGGT CTGCGATTCC GACTCGTCCA ACATCAATAC AACCTATTAA TTTCCCCTCG | |
| TCAAAAATAA GGTTATCAAG TGAGAAATCA CCATGAGTGA CGACTGAATC CGGTGAGAAT | |
| GGCAAAAGCT TATGCATTTC TTTCCAGACT TGTTCAACAG GCCAGCCATT ACGCTCGTCA | |
| TCAAAATCAC TCGCATCAAC CAAACCGTTA TTCATTCGTG ATTGCGCCTG AGCGAGACGA | |
| AATACGCGAT CGCTGTTAAA AGGACAATTA CAAACAGGAA TCGAATGCAA CCGGCGCAGG | |
| AACACTCCCA GCGCATCAAC AATATTTTCA CCTGAATCAG GATATTCTTC TAATACCTGG | |
| AATGCTGTTT TCCCGGGGAT CGCAGTGGTG AGTAACCATG CATCATCAGG AGTACGGATA | |
| AAATGCTTGA TGGTCGGAAG AGGCATAAAT TCCGTCAGCC AGTTTAGTCT GACCATCTCA | |
| TCTGTAACAT CATTGGCAAC GCTACCTTTG CCATGTTTCA GAAACAACTC TGGCGCATCG | |
| GGCTTCCCAT ACAATCGATA GATTGTCGCA CCTGATTGCC CGACATTATC GCGAGCCCAT | |
| TTATACCCAT ATAAATCAGC ATCCATGTTG GAATTTAATC GCGGCCTCGA GCAAGACGTT | |
| TCCCGTTGAA TATGGCTCAT AACACCCCTT GTATTACTGT TTATGTAAGC AGACAGTTTT | |
| ATTGTTCATG ATGATATATT TTTATCTTGT GCAATGTAAC ATCAGAGATT TTGAGACACA | |
| ACGTGGCTTT CCCCCCCCCC CCATTATTGA AGCATTTATC AGGGTTATTG TCTCATGAGC | |
| GGATACATAT TTGAATGTAT TTAGAAAAAT AAACAAATAG GGGTTCCGCG CACATTTCCC | |
| CGAAAAGTGC CACCTGACGT CTAAGAAACC ATTATTATCA TGACATTAAC CTATAAAAAT | |
| AGGCGTATCA CGAGGCCCTT TCGTC. | |

The underlined nucleotides of SEQ ID NO:9 represent the SfiI site introduced into the Kpn 1 site of V1Jneo while the underlined/italicized nucleotides represent the human tPA leader sequence.

V1R—Vaccine vector V1R was constructed to obtain a minimum-sized vaccine vector without unneeded DNA sequences, which still retained the overall optimized heterologous gene expression characteristics and high plasmid yields that V1J and V1Jns afford. It was determined that (1) regions within the pUC backbone comprising the E. coli origin of replication could be removed without affecting plasmid yield from bacteria; (2) the 3′-region of the kan^{r} gene following the kanamycin open reading frame could be removed if a bacterial terminator was inserted in its place; and, (3) ˜300 bp from the 3′-half of the BGH terminator could be removed without affecting its regulatory function (following the original KpnI restriction enzyme site within the BGH element). V1R was constructed by using PCR to synthesize three segments of DNA from V1Jns representing the CMVintA promoter/BGH terminator, origin of replication, and kanamycin resistance elements, respectively. Restriction enzymes unique for each segment were added to each segment end using the PCR oligomers: SspI and XhoI for CMVintA/BGH; EcoRV and BamHI for the kan^{r} gene; and, BclI and SalI for the ori^{r}. These enzyme sites were chosen because they allow directional ligation of each of the PCR-derived DNA segments with subsequent loss of each site: EcoRV and SspI leave blunt-ended DNAs which are compatible for ligation while BamHI and BclI leave complementary overhangs as do SalI and XhoI. After obtaining these segments by PCR each segment was digested with the appropriate restriction enzymes indicated above and then ligated together in a single reaction mixture containing all three DNA segments. The 5′-end of the ori^{r} was designed to include the T2 rho independent terminator sequence that is normally found in this region so that it could provide termination information for the kanamycin resistance gene. The ligated product was confirmed by restriction enzyme digestion (>8 enzymes) as well as by DNA sequencing of the ligation junctions. DNA plasmid yields and heterologous expression using viral genes within V1R appear similar to V1Jns. The net reduction in vector size achieved was 1346 bp (V1Jns=4.86 kb; V1R=3.52 kb). PCR oligomer sequences used to synthesize V1R (restriction enzyme sites are underlined and identified in brackets following sequence) are as follows: (1) 5′-GGTACAAATATTGGCTATTGGC CATTGCATACG-3′ (SEQ ID NO:20) [SspI]; (2) 5′-CCACATCTCGAGGAA CCGGGTCAATTCTTCAGCACC-3′(SEQ ID NO:21) [XhoI] (for CMVintA/BGH segment); (3) 5′-GGTACAGATATCGGAAAGCCACGTTGTG TCTCAAAATC-3′ (SEQ ID NO:22) [EcoRV]; (4) 5′-CACATGGATCCGTAATGCTCTGCCAGTGT TACAACC-3′ (SEQ ID NO:23) [BamHI], (for kanamycin resistance gene segment) (5) 5′-GGTACATG ATCACGTAGAAAAGATCAAAGGATCTTCTTG-3′ (SEQ ID NO:24) [BclI]; (6) 5′-CCACATGTCGACCCGTAAAAAGGCCGCGTTGCTGG-3′ (SEQ ID NO:25): [SalI], (for E. coli origin of replication).

The nucleotide sequence of vector V1R is as follows:

| | | | | | | |
|---|---|---|---|---|---|---|
| TCGCGCGTTT | CGGTGATGAC | GGTGAAAACC | TCTGACACAT | GCAGCTCCCG | GAGACGGTCA | (SEQ ID NO:26) |
| CAGCTTGTCT | GTAAGCGGAT | GCCGGGAGCA | GACAAGCCCG | TCAGGGCGCG | TCAGCCGGTG | |
| TTGGCGGGTG | TCGGGGCTGG | CTTAACTATG | CGGCATCAGA | GCAGATTGTA | CTGAGAGTGC | |
| ACCATATGCG | GTGTGAAATA | CCGCACAGAT | GCGTAAGGAG | AAAATACCGC | ATCAGATTGG | |
| CTATTGGCCA | TTGCATACGT | TGTATCCATA | TCATAATATG | TACATTTATA | TTGGCTCATG | |
| TCCAACATTA | CCGCCATGTT | GACATTGATT | ATTGACTAGT | TATTAATAGT | AATCAATTAC | |
| GGGGTCATTA | GTTCATAGCC | CATATATGGA | GTTCCCCGTT | ACATAACTTA | CGGTAAATGG | |
| CCCGCCTGGC | TGACCGCCCA | ACGACCCCCG | CCCATTGACG | TCAATAATGA | CGTATGTTCC | |
| CATAGTAACG | CCAATAGGGA | CTTTCCATTG | ACGTCAATGG | GTGGAGTATT | TACGGTAAAC | |
| TGCCCACTTG | GCAGTACATC | AAGTGTATCA | TATGCCAAGT | ACGCCCCCTA | TTGACGTCAA | |
| TGACGGTAAA | TGCCCCGCCT | GGCATTATGC | CCAGTACATG | ACCTTATGGG | ACTTTCCTAC | |
| TTGGCAGTAC | ATCTACGTAT | TAGTCATCGC | TATTACCATG | GTGATGCGGT | TTTGGCAGTA | |
| CATCAATGGG | CGTGGATAGC | CGTTTGACTC | ACGGGGATTT | CCAAGTCTCC | ACCCCATTGA | |
| CGTCAATGGG | AGTTTGTTTT | GGCACCAAAA | TCAACGGGAC | TTTCCAAAAT | GTCGTAACAA | |
| CTCCGCCCCA | TTGACGCAAA | TGGGCGGTAG | GCGTGTACGG | TGGGAGGTCT | ATATAAGCAG | |
| ACCTCGTTTA | GTGAACCGTC | AGATCGCCTG | GAGACGCCAT | CCACGCTGTT | TTGACCTCCA | |
| TAGAAGACAC | CGGGACCGAT | CCAGCCTCCG | CGGCCGGGAA | CGGTGCATTG | GAACGCGCAT | |
| TCCCCGTGCC | AAGAGTGACG | TAAGTACCGC | CTATAGAGTC | TATAGGCCCA | CCCCCTTGGC | |
| TTCTTATGCA | TGCTATACTG | TTTTTGGCTT | GGGGTCTATA | CACCCCCGCT | TCCTCATGTT | |
| ATAGGTGATG | GTATAGCTTA | GCCTATAGGT | GTGGGTTATT | GACCATTATT | GACCACTCCC | |
| CTATTGGTGA | CGATACTTTC | CATTACTAAT | CCATAACATG | GCTCTTTGCC | ACAACTCTCT | |
| TTATTGGCTA | TATGCCAATA | CACTGTCCTT | CAGAGACTGA | CACGGACTCT | GTATTTTTAC | |
| AGGATGGGGT | CTCATTTATT | ATTTACAAAT | TCACATATAC | AACACCACCG | TCCCCAGTGC | |
| CCGCAGTTTT | TATTAAACAT | AACGTGGGAT | CTCCACGCGA | ATCTCGGGTA | CGTGTTCCGC | |
| ACATGGGCTC | TTCTCCGGTA | GCGGCGGAGC | TTCTACATCC | GAGCCCTGCT | CCCATGCCTC | |
| CAGCGACTCA | TGGTCGCTCG | GCAGCTCCTT | GCTCCTAACA | GTGGAGGCCA | GACTTAGGCA | |
| CAGCACGATG | CCCACCACCA | CCAGTGTGCC | GCACAACGCC | GTCGCGGTAG | GGTATGTGTC | |
| TGAAAATGAG | CTCGGGGAGC | GGGCTTGCAC | CGCTGACGCA | TTTGCAACAC | TTAAGGCAGC | |
| GGCAGAAGAA | GATGCAGGCA | GCTGAGTTGT | TGTGTTCTGA | TAAGAGTCAG | AGGTAACTCC | |
| CGTTGCGGTG | CTGTTAACGG | TGGAGGGCAG | TGTAGTCTGA | GCAGTACTCG | TTGCTGCCGC | |
| GCGCGCCACC | AGACATAATA | GCTGACAGAC | TAACAGACTG | TTCCTTTCCA | TGGGTCTTTT | |
| CTGCAGTCAC | CGTCCTTAGA | TCTGCTGTGC | CTTCTAGTTG | CCAGCCATCT | GTTGTTTGCC | |
| CCTCCCCCGT | GCCTTCCTTG | ACCCTGGAAG | GTGCCACTCC | CACTGTCCTT | TCCTAATAAA | |
| ATGAGGAAAT | TGCATCGCAT | TGTCTGACTA | GGTGTCATTC | TATTCTGGGG | GGTGGGGTGG | |
| GGCAGCACAG | CAAGGGGGAC | GATTGGGAAG | ACAATAGCAG | GCATGCTGGG | GATGCGGTGG | |
| GCTCTATGGG | TACCCAGGTG | CTGAAGAATT | GACCCGGTTC | CTCCTGGGCC | AGAAAGAAGC | |
| AGGCACATCC | CCTTCTCTGT | GACACACCCT | GTCCACGCCC | CTGGTTCTTA | GTTCCAGCCC | |
| CACTCATAGG | ACACTCATAG | CTCAGGAGGG | CTCCGCCTTC | AATCCCACCC | GCTAAAGTAC | |
| TTGCAGCCGT | CTCTCCCTCC | CTCATCAGCC | CACCAAACCA | AACCTAGCCT | CCAAGAGTGG | |
| GAAGAAATTA | AACCAAGATA | GGCTATTAAG | TGCAGAGGGA | CAGAAAATGC | CTCCAACATG | |
| TGAGGAAGTA | ATGAGAGAAA | TCATAGAATT | TCTTCCGCTT | CCTCGCTCAC | TGACTCGCTG | |
| CGCTCGGTCG | TTCGGCTGCG | GCGAGCGGTA | TCAGCTCACT | CAAAGGCGGT | AATACGGTTA | |
| TCCACAGAAT | CAGGGGATAA | CGCAGGAAAG | AACATGTGAG | CAAAAGGCCA | GCAAAAGGCC | |
| AGGAACCGTA | AAAAGGCCGC | GTTGCTGGCG | TTTTTCCATA | GGCTCCGCCC | CCCTCACGAG | |
| CATCACAAAA | ATCGACGCTC | AAGTCAGAGG | TGGCGAAACC | CGACAGGACT | ATAAAGATAC | |
| CAGGCGTTTC | CCCCTGGAAG | CTCCCTCGTG | CGCTCTCCTG | TTCCGACCCT | GCCGCTTACC | |
| GGATACCTGT | CCGCCTTTCT | CCCTTCGGGA | ACCGTGGCGC | TTTCTCAATG | CTCACGCTGT | |
| AGGTATCTCA | GTTCGGTGTA | GGTCGTTCGC | TCCAAGCTGG | GCTGTGTGCA | CGAACCCCCC | |
| GTTCAGCCCG | ACCGCTGCGC | CTTATCCGGT | AACTATCGTC | TTGAGTCCAA | CCCGGTAAGA | |
| CACGACTTAT | CGCCACTGGC | AGCAGCCACT | GGTAACAGGA | TTAGCAGAGC | GAGGTATCTA | |
| GGCGGTGCTA | CAGAGTTCTT | CAAGTGGTGC | CCTAACTACG | GCTACACTAG | AAGGACAGTA | |
| TTTGGTATCT | GCGCTCTGCT | CAAGCCAGTT | ACCTTCGGAA | AAAGAGTTGG | TAGCTCTTGA | |
| TCCGGCAAAC | AAACCACCGC | TGGTAGCGGT | GGTTTTTTTG | TTTGCAAGCA | GCAGATTACG | |
| CGCAGAAAAA | AAGGATCTCA | AGAAGATCCT | TTGATCTTTT | CTACGGGGTC | TGACCCTCAG | |
| TGGAACGAAA | ACTCACGTTA | AGGGATTTTG | GTCATGAGAT | TATCAAAAAC | GATCTTCACC | |
| TAGATCCTTT | TAAATTAAAA | ATGAAGTTTT | AAATCAATCT | AAAGTATATA | TGAGTAAACT | |
| TGGTCTGACA | GTTACCAATG | CTTAATCAGT | GAGGCACCTA | TCTCAGCGAT | CTGTCTATTT | |
| CGTTCATCCA | TAGTTGCCTG | ACTCCCCGGG | GGGGGGCCGC | TGAGGTCTGC | CTCGTGAAGA | |
| AGGTGTTGCT | GACTCATACC | AGGCCTGAAT | CGCCCCATCA | TCCAGCCAGA | AAGTGAGGGA | |
| GCCACGGTTG | ATGAGAGCTT | TGTTGTAGGT | GGACCAGTTG | GTGATTTTGA | ACTTTTGCTT | |
| TGCCACGGAA | CGGTCTGCGT | TGTCGGGAAG | ATGCGTGATC | TGATCCTTCA | ACTCAGCAAA | |
| AGTTCGATTT | ATTCAACAAA | CCCGCCGTCC | CGTCAAGTCA | GCGTAATGCT | CTGCCAGTGT | |
| TACAACCAAT | TAACCAATTC | TGATTAGAAA | AACTCATCGA | GCATCAAATG | AAACTCCAAT | |
| TTATTCATAT | CAGGATTATC | AATACCATAT | TTTTGAAAAA | GCCGTTTCTG | TAATGAACGA | |
| GAAAACTCAC | CGAGGCAGTT | CCATAGGATG | GCAAGATCCT | GGTATCGGTC | TGCGATTCCG | |
| ACTCGTCCAA | CATCAATACA | ACCTATTAAT | TTCCCCTCGT | CAAAAATAAG | GTTATCAAGT | |
| GAGAAATCAC | CATGAGTGAC | GACTGAATCC | GGTGAGAATG | GCAAAAGCTT | ATGCATTTCT | |
| TTCCAGACTT | GTTCAACAGG | CCAGCCATTA | CGCTCGTCAT | CAAAATCACT | CGCATCAACC | |
| AAACCGTTAT | TCATTCGTGA | TTGCGCCTGA | GCGAGACGAA | ATACGCGATC | GCTGTTAAAA | |
| GGACAATTAC | AAACAGGAAT | CGAATGCAAC | CGGCGCAGGA | ACACTGCCAG | CGCATCAACA | |
| ATATTTTCAC | CTGAATCAGG | ATATTCTTCT | AATACCTGGA | ATGCTGTTTT | CCCGGGGATC | |
| GCAGTGGTGA | GTAACCATGC | ATCATCAGGA | GTACGCATAA | AATGCTTGAT | GGTCGGAAGA | |
| GGCATAAATT | CCGTCAGCCA | GTTTAGTCTG | ACCATCTCAT | CTGTAACATC | ATTGGCAACG | |
| CTACCTTTGC | CATGTTTCAG | AAACAACTCT | GGCGCATCGG | GCTTCCCATA | CAATCGATAG | |
| ATTGTCGCAC | CTGATTGCCC | GACATTATCG | CGAGCCCATT | TATACCCATA | TAAATCAGCA | |
| TCCATGTTGG | AATTTAATCC | CGGCCTCGAG | CAAGACGTTT | CCCGTTGAAT | ATGGCTCATA | |
| ACACCCCTTG | TATTACTGTT | TATGTAAGCA | GACAGTTTTA | TTGTTCATGA | TGATATATTT | |
| TTATCTTGTG | CAATGTAACA | TCAGAGATTT | TGAGACACAA | CGTGGCTTTC | CCCCCCCCCC | |
| CATTATTGAA | CCATTTATCA | GGGTTATTGT | CTCATGAGCG | GATACATATT | TGAATGTATT | |
| TAGAAAAATA | AACAAATAGG | GGTTCCGCGC | ACATTTCCCC | GAAAAGTGCC | ACCTGACGTC | |
| TAAGAAACCA | TTATTATCAT | GACATTAACC | TATAAAAATA | GGCGTATCAC | GAGGCCCTTT | |
| CGTC. | | | | | | |

### EXAMPLE 2

### Codon Optimized HIV-1 Nef and HIV-1 Nef Derivatives as DNA Vector Vaccines

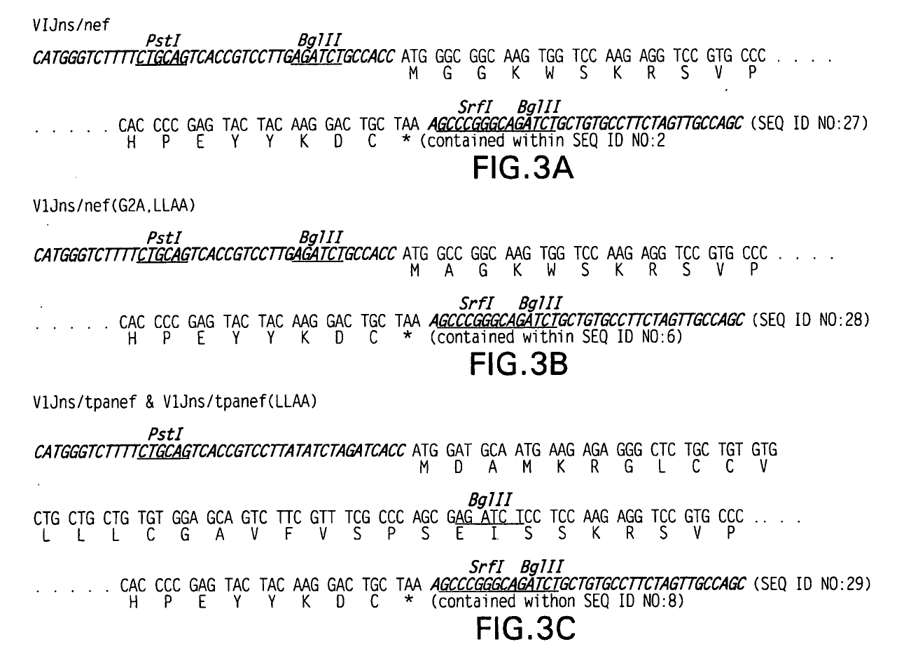

HIV-1 Nef Vaccine Vectors—Codon optimized nef gene coding for wt Nef protein of HIV-1 jrfl isolate was assembled from complementary, overlapping synthetic oligonucleotides by polymerase chain reaction (PCR). The PCR primers used were designed in such that a BglII site was included in the extension of 5′ primer and an SrfI site and a BglII site in the extension of 3′ primer. The PCR product was digested with BglII and cloned into BglII site of a human cytomeglovirus early promoter-based expression vector, V1Jns (FIG. 1A). The proper orientation of nef fragment in the context of the expression cassette was determined by asymmetric restriction mapping. The resultant plasmid is V1Jns/nef. The 5′ and 3′ nucleotide sequence junctions of codon optimized V1Jns/nef are shown in FIG. 3A.

The mutant nef (G2A,LLAA) was also made from synthetic oligonucleotides. To assist in cloning, a PstI site and an SrfI site were included in the extensions of 5′ and 3′ PCR primers, respectively. The PCR product was digested with PstI and SrfI, and cloned into the PstI and SrfI sites of V1Jns/nef, replacing the original nef with nef(G2A,LLAA) fragment. This resulted in V1Jns/nef(G2A,LLAA). The 5′ and 3′ nucleotide sequence junctions of codon optimized V1Jns/nef (G2A,LLAA) are shown in FIG. 3B.

To construct the expression vector containing human tissue plasminogen activator leader peptide and the nef fusion gene, i.e., V1Jns/tPAnef, a truncated nef gene fragment, lacking the coding sequence for the five amino terminal residues, was first amplified by PCR using V1Jns/nef as template. Both 5′ and 3′ PCR primers used in this reaction contained a BglII extension. The PCR amplified fragment was then digested with BglII and cloned into BglII site of the expression vector, V1Jns/tpa (FIG. 1B). The ligation of the 3′ end of tpa leader peptide coding sequence to the 5′ end of the nef PCR product restored the BglII site and yielded an in-frame fusion of the two genes. The 5′ and 3′ nucleotide sequence junctions of codon optimized V1Jns/tPAnef are shown in FIG. 3C.

Construction of V1Jns/tpanef(LLAA) was carried out by replacing the Bsu36-SacI fragment of V1Jns/tpanef, which contains the 3′ half of the nef gene and part of the vector backbone, with the Bsu36-SacII fragment from V1Jns/nef(G2A,LLAA). The 5′ and 3′ nucleotide sequence junctions of codon optimized V1Jns/tpanef (LLAA) are shown in FIG. 3C.

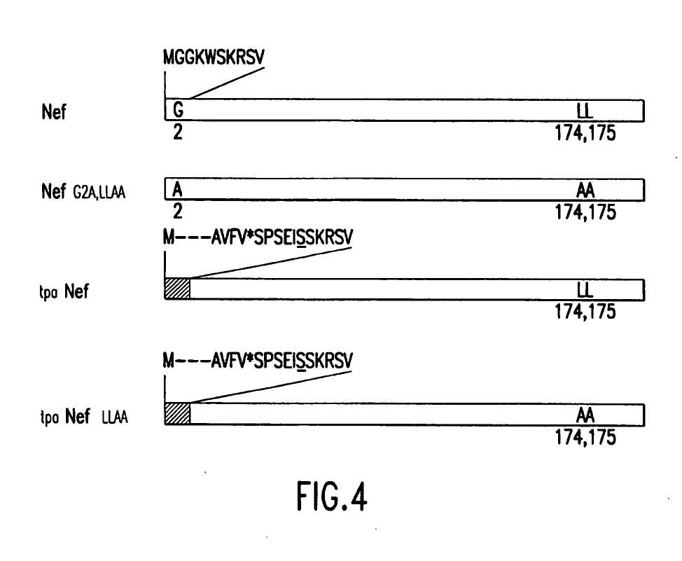

All the nef constructs were verified by sequencing. The amino acid junctions of these constructs is shown schematically in FIG. 4.

Transfection and protein expression—293 cells (adenovirus transformed human embryonic kidney cell line 293) grown at approximately 30% confluence in minimum essential medium (MEM; GIBCO, Grand Island, Md.) supplemented with 10% fetal bovine serum (FBS; GIBCO) in a 100 mm culture dish, were transfected with 4 ug gag expression vector, V1Jns/gag, or a mixture of 4 ug gag expression vector and 4 ug nef expression vector by Lipofectin following manufacture's protocol (GIBCO). Twelve hours post-transfection, cells were washed once with 10 ml of serum-free medium, Opti-MEM I (GIBCO) and replenished with 5 ml of Opti-MEM. Following an additional 60 hr incubation, culture supernatants and cells were collected separately and used for Western blot analysis.

Western blot analysis—Fifty microliter of samples were separated on a 10% SDS-polyacrylamide gel (SDS-PAGE) under reducing conditions. The proteins were blotted onto a piece of PVDF membrane, and reacted to a mixture of gag mAb (#18; Intracel, Cambridge, Mass.) and Nef mAbs (aa64-68, aa195-201; Advanced Biotechnologies, Columbia, Md.), both at 1:2000 dilution, and horseradish peroxidase (HRP)-conjugated goat anti-rabbit IgG (Zymed, San Francisco, Calif.). The protein bands were visualized by ECL Western blotting detection reagents, according to the manufacture's protocol (Amersham, Arlington Heights, Ill.).

Enzyme-linked immunosorbent assay (ELISA)-96-well Immulon II, round-bottom plates were coated with 50 ul of Nef protein at the concentration of 2 ug/ml in bicarbonate buffer, pH 9.8., per well at 4° C. overnight. Plates were washed three times with PBS containing 0.05% Tween-20 (PBST), and blocked with 5% skim milk in PBST (milk-PBST) at 24° C. for 2 hr, and then incubated with serial dilutions of testing samples in milk-PBST at 24° C. for 2 hr. Plates were washed with PBST three times, and added with 50 ul of HRP-conjugated goat anti-mouse IgG (Zymed) per well and incubated at 24° C. for 1 hr. This was followed by three washes, and the addition of 100 ul of 1 mg/ml ABTS [(2,2′-amino-di-(3-ethylbenzthiozoline sulfonate)] (KPL, Gaithersburg, Md.) per well. After 1 hr at 24° C., plates were read at a wavelength of 405 nm using an ELISA plate reader.

Enzyme-linked spot assay (Elispot)—Nitrocellulose membrane-backed 96 well plates (MSHA plates; Millipore, Bedford, Mass.) were coated with 50 ul of rat anti-mouse IFN-gamma mAb, capture antibody, (R4-6A2; PharMingen, San Diego, Calif.) at a concentration of 5 ug/ml in PBS per well at 4° C. overnight. Plates were washed three times with PBST and blocked with 10% FBS in RPMI-1640 (FBS-RPMI) at 37° C. in a CO2 incubator for 2 to 4 hrs. Splenocytes were suspended in RPMI-1640 with 10% FBS at 4×10⁶ cells per ml. 100 ul cells were added to each well and plates were incubated at 37° C. for 20 hrs. Each sample was tested in triplicate wells. After incubation, plates were rinsed briefly with distilled water and washed three times with PBST. Fifty ul of biotinylated rat anti-mouse IFN-γ mAb, detecting antibody (XMG1.2; PharMingen), diluted in 1% BSA in PBST at a concentration of 2 ug/ml was then added to each well. Plates were incubated at 24° C. for 2 hr, followed by washes with PBST. Fifty ul of streptavidin-conjugated alkaline phosphatase (KPL) at a dilution of 1:1000 in FBS-RPMI was added to each well. The plates were incubated at 24 C for an additional one hr. Following extensive wash with BPST, 100 ul BCIT/NBT substrate (KPL) was added for 15 min, and color reaction was stopped by washing the plate with tap water. Plates were air-dried and spots were countered using a dissection microscope.

Cytotoxic T cell (CTL) assay—Splenocytes from immunized mouse were co-cultured with syngenic peptide-pulsed, irradiated naive splenocytes for 7 days. EL-4 cells were incubated at 37° C. for 1 hr with or without 20 ug/ml of a designated peptide in the presence of sodium 51Cr-chromate and used as target cells. For the assay, 10⁴ target cells were added to a 96-well plate along with different numbers of splenocytes cells. Plates were incubated at 37° C. for 4 hr. After incubation, supernatants were collected and counted in a Wallac gamma-counter. Specific lysis was calculated as ([experimental release—spontaneous release]/maximum release—spontaneous release])×100%. Spontaneous release was determined by incubating target cells in medium alone, and maximum release was determined by incubating target cells in 2.5% TritonX-100. The assay was performed with triplicate samples.

Animal experiments—Female mice (Charles River Laboratories, Wilmington, Mass.), 6 to 10 weeks old, were injected in quadriceps with 100 ul of DNA in PBS. Two weeks after immunization, spleens from individual mice were collected and used for CTL and Elispot assays.

Results (DNA Vector Vaccine Construction)—The exemplified Nef protein sequence is based on HIV-1 clade B jrfl isolate. A codon-optimized nef gene was chosen for vaccine construction and for use as the parental gene for other exemplified constructs. FIG. 2A-B show the comparison of coding sequence of wt nef(jrfl) and the codon optimized nef(jrfl). Two forms of myristylation site mutations were constructed; one contains a Gly2Ala change and the other a human tissue plasminogen activator (tpa) leader sequence was fused to sixth residue, Ser, of Nef (tpanef). The dileucine motif mutation was made by introducing both Leu174Ala and Leu175Ala changes. FIG. 4 shows the schematic depiction of the Nef and Nef mutants. For in vitro expression and in vivo immunogenicity studies, the nef genes were cloned into expression vector, V1Jns. The resultant plasmids containing wt nef, tpanef, tpanef with dileucine motif mutation, and nef mutant with the Gly2Ala myristylation site and dileucine motif mutations were named as V1Jns/nef, V1Jns/tpanef, V1Jns/tpanef(LLAA) and V1Jns/(G2A,LLAA), respectively.

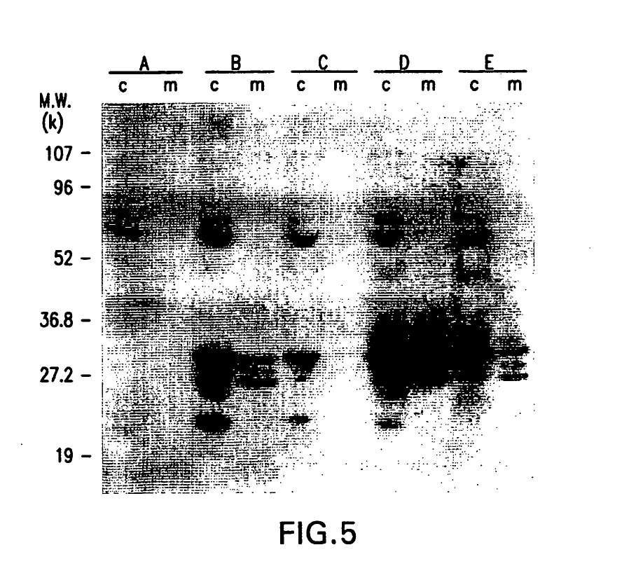

Results—Expression and Western blotting analysis—To evaluate the expression of the codon optimized nef constructs, adenovirus-transformed human kidney 293 cells were cotransfected with individual nef plasmids and a gag expression vector, V1Jns/gag. 72 hours post transfection, cells and medium were collected separately and analyzed by Western blotting, using both Nef- and Gag-specific mAbs. The results are shown in FIG. 5. Cells transfected with V1Jns/gag only revealed a single distinct band of approximately 55 Kd, whereas the cells cotransfected with gag and nef plasmids revealed, in addition to the 55 Kd band, a major 30 Kd band and several minor bands. This pattern is consistent with that the 55 Kd species represents Gag polypeptide and the 30 Kd and other minor species are the Nef-related products. Therefore, all the nef constructs were expressed in the transfected cells. When measured against the relatively constant Gag signal as a reference, four nef genes seem to be expressed at different levels, with the following descending order, tpanef, nef, tpanef(LLAA) and nef(G2A, LLAA). With the exception of nef(G2A,LLAA), products of nef, tpanef, tpanef(LLAA) could be detected in both cellular and medium fractions.

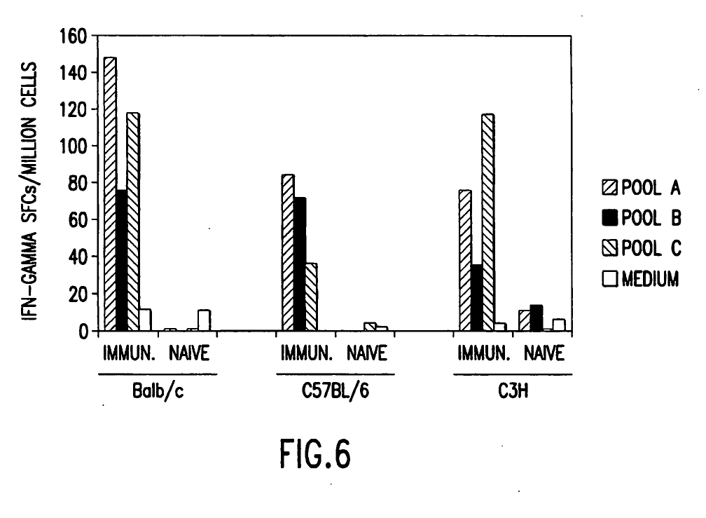

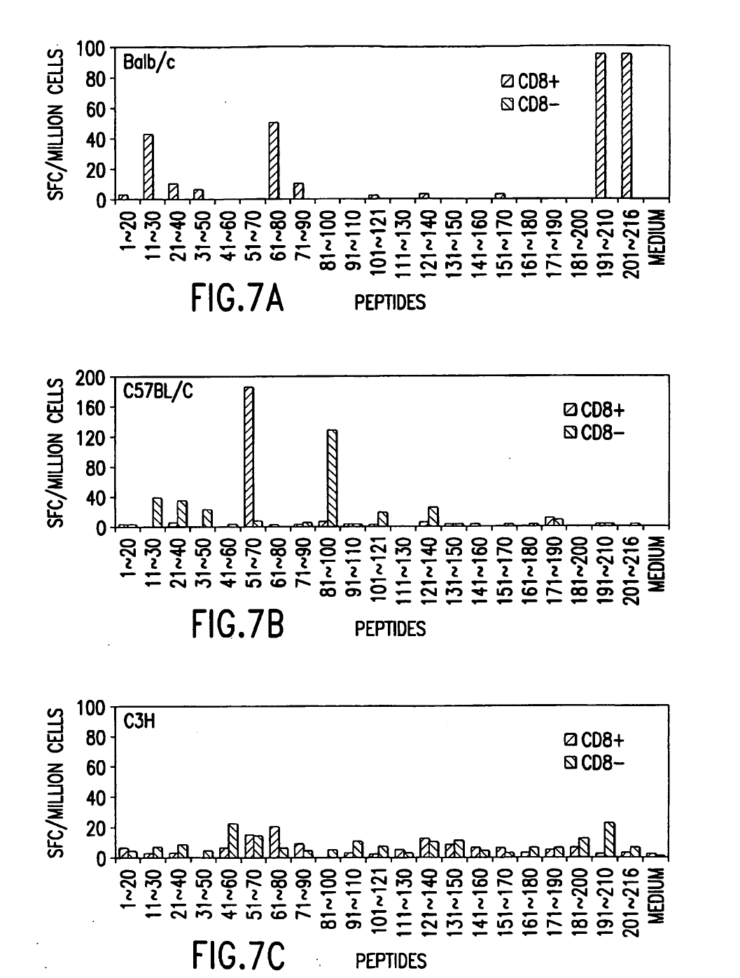

Mapping of Nef-specific CD8 and CD4 epitopes in mice—There was no information available with respect to the properties of Nef(jrfl) in eliciting cell-mediated immune responses in mice. Therefore, to characterize immunogenicity of Nef and Nef mutants exemplified herein, CD8 and CD4 epitopes were mapped. An overlapping set of overlapping nef peptides that encompass the entire 216 aa Nef polypeptide were generated. A total 21 peptides were made, which include twenty 20mers and one 16mer. Three strains of mice, Balb/c, C3H and C57BL/6, were immunized with plasmid V1Jns/Nef; splenocytes from immunized and naive mice were isolated and assessed for Nef specific INF-gamma secreting cells (SFC) by the Elispot assay. FIG. 6 shows where Elispot assays were performed against separate pools of the Nef peptides. All three strains of immunized mice responded to the Nef plasmid immunization; each developed positive Nef peptide-specific INF-γ SFCs. Based on this, further studies were carried out with fractionated CD8 and CD4 cells against individual peptides. The results are shown in FIG. 7A-C. In Balb/c mice (FIG. 7A), four Nef peptides, namely, aa11-30, aa61-80, aa191-210 and aa200-216, were found to be able to induce significant numbers of CD4 SFCs. In C57BL/6 mice (FIG. 7B), only one peptide, ie., aa81-100, elicited significant numbers of CD4 SFCs. Compared to Balb/c and C57B/6 mice, C3H mice (FIG. 7C) showed no dominant CD4 SFC responses with particular peptides; instead, there were modest number of SFCs in response to an array of peptides, including aa21-40, aa3′-50, aa121-140 aa13′-150, aa181-200 and aa191-210. With respect to CD8 cells, significant SFC responses were detected with a single peptide, ie., aa51-70, in C57BL/6 mice only.

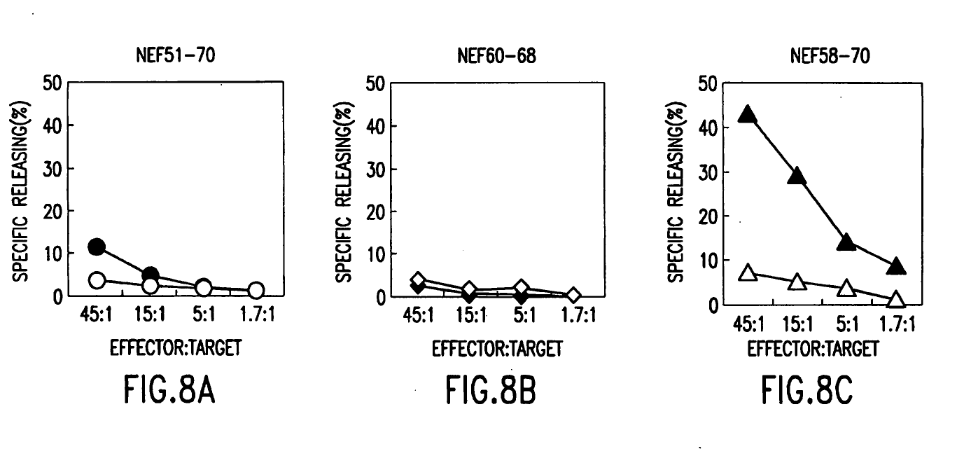

The results from Elispot assay suggested that Nef peptide aa51-70 contained an H-2b restricted CD8 cell epitope. In order to ascertain whether this CD8 epitope also represents the cytotoxic T cell (CTL) epitope, a conventional CTL assay was carried out. The peptide aa51-70 (FIG. 8A) induced low level of specific killings only. Peptides longer than 9 amino acids of a typical CTL epitope often have lower binding affinity to MHC class I molecule. It was contemplated that the low specific killings observed with peptide aa51-70 could be potentially resulted from the low binding affinity of this 20 amino acid peptide. Therefore, two shortened peptides, namely, aa60-68 and aa58-70, were synthesized and tested in CTL assays. While the peptide aa60-68 failed to elicit any specific killings (FIG. 8B), the peptide aa58-70 exhibited a drastic increase of specific killing as compared to its longer counterpart, peptide aa61-80 (FIG. 8C). For example, the percentage of specific killings induced by peptide aa58-70 at an effector/target ratio of 5 to 1 was comparable to that induced by peptide aa51-80 at an effector/target ratio of 45. Thus, between peptide aa58-70 and peptide aa51-70, the former was almost ten-fold more effective in terms of inducing Nef-specific killing. The results from CTL assay therefore confirmed that the CD8 epitope detected by the Elispot assay was indeed a CTL epitope. To further map the minimum amino acid sequence for the Nef CTL epitope, additional 5 peptides were synthesized and analyzed by Elispot assay, which mapped the CTL epitope to Nef aa58-66, as shown in Table 1.

TABLE 1

| Nef peptides** | | INF-γ SFC*/10⁶ splenocytes |
|---|---|---|
| Nef58-70 | TAATNADCAWLEA | 85 |
| Nef59-69 | AATNADCAWLE | 1 |
| Nef58-68 | TAATNADCAWL | 69 |
| Nef58-67 | TAATNADCAW | 66 |
| Nef58-66 | TAATNADCA | 92 |
| Medium | | 1 |

*Average of duplicate samples.
**Amino acid sequence of all peptides contained within SEQ ID NO: 2.

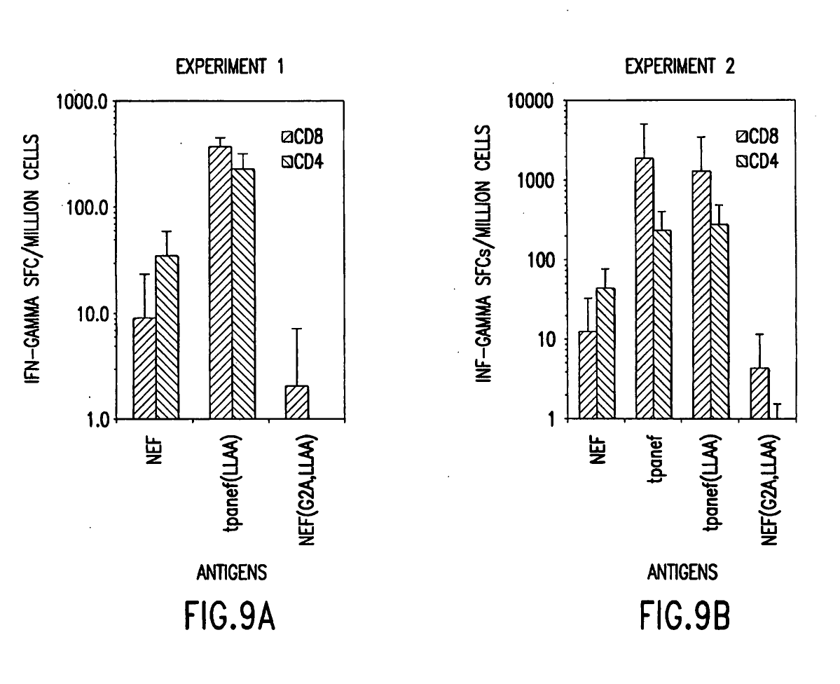

Results (Evaluation of Immunogenicity of nef Mutants in Mice)—Having identified H-2b restricted CTL and CD4 cell epitopes, the immunogenicity of the different codon optimized nef constructs in C57BL/6 mice was examined. This was performed in two separate experiments with identical immunization regimens. The first experiment involved nef, tpanef(LLAA) and nef(G2A,LLAA) and the second experiment involved nef, tpanef, tpanef(LLAA) and nef(G2A,LLAA). Mice were immunized with plasmids containing these respective codon optimized nef genes. Two weeks post immunization, splenocytes from individual mice were isolated and analyzed by Elispot assay for Nef-specific CD8 and CD4 IFN-gamma SFCs using Nef peptide aa58-66 and aa81-100, respectively. The results are shown in FIG. 9A-B. In the experiment 1 (FIG. 9A), among the three groups tested, the mice receiving the codon optimized tpanef(LLAA) construct developed the highest CD8 and CD4 cell responses; comparing between tpanef(LLAA) and the nef, the former elicited about 40-fold higher CD8 SFCs and 10-fold higher CD4 SFCs. In contrast to tpanef(LLAA), nef(G2A,LLAA) mutant was poorly immunogenic; mice receiving this mutant had barely detectable CD8 and CD4 SFCS, under conditions tested. Similar response profiles between the three mutants were also observed in the experiment 2 (FIG. 9B), except that the overall CD8 response of mice receiving tpanef(LLAA) was approximately 10-folder higher in experiment 2 than that observed in experiment 1. The tPAnef mutant showed comparable responses as that of tpanef(LLAA). The results therefore showed that both codon optimized tpanef and tpanef(LLAA) had significantly enhanced immunogenicity.

Results (Evaluation of Immunogenicity of nef Mutants in Rhesus Monkeys)—Monkeys were immunized with 5 mg of indicated codon optimized plasmids at week 0, 4, and 8. Four weeks after each immunization, peripheral blood mononuclear cells were collected and tested for Nef-specific INF-gamma secreting cells as described for the mice studies in this Example section. The results are shown in Table 2. As with the mouse study, tpanef(LLAA) shows significantly enhanced immunogenicity when compared to tPAnef.

TABLE 2
Nef specific INF-gamma secreting cells/million PBMC

| | Animal | Week 0 | | Week 4 | | Week 8 | | Week 12 | |
|---|---|---|---|---|---|---|---|---|---|
| Vaccine | No. | Medium | nef | Medium | nef | Medium | nef | Medium | nef |
| VIJns- | 1 | 74 | 39 | 30 | 208 | 6 | 148 | 89 | 559 |
| TpaNef | 2 | 1 | 3 | 28 | 45 | 13 | 44 | 13 | 146 |
| (LLAA) | 3 | 5 | 5 | 14 | 45 | 11 | 11 | 14 | 35 |
| VIJns-nef | 1 | 0 | 1 | 24 | 33 | 16 | 43 | 6 | 34 |
| | 2 | 28 | 9 | 31 | 35 | 13 | 34 | 24 | 80 |
| | 3 | 1 | 0 | 16 | 31 | 18 | 38 | 13 | 185 |
| Control | 1 | 1 | 3 | 16 | 33 | 16 | 16 | 18 | 13 |

Monkeys were immunized with 5 mg of indicated plasmids at week 0, 4 and 8.
Four weeks after each immunization, peripheral blood mononuclear cells were collected and tested for the Nef-specific IFN-gamma secreting cells.

A codon-optimized nef gene coding for HIV-1 jrfl isolate Nef polypeptide was synthesized. The resultant synthetic nef gene was well expressed in the in vitro transfected cells. Using this synthetic gene as parental molecule, nef mutants involving myristylation site and dileucine motif mutations were constructed. Two forms of myristylation site mutation were made, one involving a single Gly2Ala change and the other by fusing human plasminogen activator(tpa) leader peptide with the N-terminus of Nef polypeptide. The dileucine motif mutation was generated by Leu174Ala and Leu175Ala changes. The resultant nef constructs were named as nef, tpanef, tpanef(LLAA) and nef(G2A,LLAA). The addition of tpa leader peptide sequence resulted in significantly increased expression of the nef gene in vitro; in contrast, either Gly2Ala mutation or dileucine mutation reduced the nef gene expression. In an effort to characterize immunogenicity of nef and nef mutants, experiments were carried out to map nef CTL and Th epitopes in mice. A single CTL epitope and a dominant Th epitope, both restricted by H-2b, were identified. Consequently, C57BL/6 mice were immunized with different nef constructs by DNA immunization means, and splenocytes from immunized mice were determined for Nef-specific CTL and Th responses using Elisopt assay and the defined T cell epitopes. The results showed that tpanef and tpanef(LLAA) were significantly more immunogenic than nef in terms of eliciting both CTL and Th responses.

Therefore, these aforementioned polynucleotides, when directly introduced into a vertebrate in vivo, including mammals such as primates and humans, should express the respective HIV-1 Nef protein within the animal and in turn induce at least a cytotoxic T lymphocyte (CTL) response within the host to the expressed Nef antigen.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A pharmaceutically acceptable DNA vaccine, which comprises:
(a) a DNA expression vector; and,
(b) a DNA molecule containing a codon optimized open reading frame encoding a HIV Nef protein or immunogenic Nef derivative thereof, wherein upon administration of the DNA vaccine to a host the Nef protein or immunogenic Nef derivative is expressed and generates an immune response which provides a substantial level of protection against HIV-1 infection.

2. A DNA vaccine of claim 1 wherein the DNA molecule encodes wild type Nef.

3. A DNA vaccine of claim 2 wherein the DNA molecule contains the nucleotide sequence as set forth in SEQ ID NO:1.

4. The DNA vaccine of claim 3 which is V1Jns-opt nef (jrfl).

5. A DNA vaccine of claim 2 wherein the DNA molecule expresses a wild type Nef protein which comprises the amino acid sequence as set forth in SEQ ID NO:2.

6. A DNA vaccine of claim 1 wherein the DNA molecule encodes an immunogenic Nef derivative which contains a leader peptide.

7. A DNA vaccine of claim 6 wherein the DNA molecule encodes an immunogenic Nef derivative which contains a human tissue plasminogen activator leader peptide.

8. A DNA vaccine of claim 7 wherein the DNA molecule contains the nucleotide sequence as set forth in SEQ ID NO:3.

9. The DNA vaccine of claim 8 which is V1Jns-opt tpanef.

10. A DNA vaccine of claim 7 wherein the DNA molecule expresses an immunogenic Nef derivative which comprises the amino acid sequence as set forth in SEQ ID NO:4.

11. A DNA vaccine of claim 6 wherein the DNA molecule encodes an immunogenic Nef derivative modified at the dileucine motif of amino acid residue 174 and amino acid residue 175.

12. A DNA vaccine of claim 11 wherein the DNA molecule encodes an immunogenic Nef derivative which contains a human tissue plasminogen activator leader peptide.

13. A DNA vaccine of claim 12 wherein the DNA molecule contains the nucleotide sequence as set forth in SEQ ID NO:7.

14. The DNA vaccine of claim 13 which is V1Jns-opt tpanef (LLAA).

15. A DNA vaccine of claim 11 wherein the DNA molecule expresses an immunogenic Nef derivative which comprises the amino acid sequence as set forth in SEQ ID NO:8.

16. A DNA vaccine of claim 11 wherein the DNA molecule encodes a Nef protein where the glycine residue of amino acid residue 2 of Nef is modified to encode for an amino acid residue other the glycine.

17. A DNA vaccine of claim 16 wherein the DNA molecule contains the nucleotide sequence as set forth in SEQ ID NO:5.

18. A DNA vaccine of claim 17 which is V1Jns-opt nef (G2A LLAA).

19. A DNA vaccine of claim 16 wherein the DNA molecule expresses an immunogenic Nef derivative which comprises the amino acid sequence as set forth in SEQ ID NO:6.

20. A DNA vaccine of claim 1 which further comprises an adjuvant.

21. A DNA vaccine of claim 20 whrerein the adjuvant is selected from the group consisting of alumunum phosphate, calcium phosphate and a non-ionic block copolymer.

22-25. (canceled)

26. A method for inducing a cell mediated immune (CTL) response against infection or disease caused by virulent strains of HIV which comprises administering into the tissue of a vertebrate host a pharmaceutically acceptable DNA vaccine composition which comprises a DNA expression vector and a DNA molecule containing a codon optimized open reading frame encoding a Nef protein or immunogenic Nef derivative thereof, wherein upon administration of the DNA vaccine to the vertebrate host the Nef protein or immunogenic Nef derivative is expressed and generates the cell-mediated immune (CTL) response.

27. The method of claim 26 wherein the vertebrate host is a human.

28. The method of claim 26 wherein the DNA vaccine is selected from the group consisting of V1Jns-opt nef (jrfl), V1Jns-opt tpanef, V1Jns-opt tpanef (LLAA), and V1Jns-opt nef (G2A LLAA).

29. (canceled)
